(19) United States
(12) Patent Application Publication
(10) Pub. No.: US 2021/0309953 A1
(43) Pub. Date: Oct. 7, 2021

(54) CELL CULTURE, TRANSPORT AND INVESTIGATION

(71) Applicant: KONIKU, INC., San Rafael, CA (US)

(72) Inventor: Oshiorenoya E. AGABI, Dublin, CA (US)

(21) Appl. No.: 17/153,747

(22) Filed: Jan. 20, 2021

Related U.S. Application Data
(63) Continuation of application No. 15/211,557, filed on Jul. 15, 2016, now abandoned.

(30) Foreign Application Priority Data
Jul. 17, 2015 (GB) 1512600.6

(51) Int. Cl. C12M 1/00; C12M 1/34

(52) U.S. Cl. CPC C12M 23/02; C12M 23/34; C12M 41/46

(57) ABSTRACT

Provided herein is a cell culture module for culturing one or more cells in an array of chambers formed therein the cell culture module. In some cases, the cell culture module may further comprise one or more sensors. In some cases, the cell culture module may further comprise one or more electrodes.

### CROSS REFERENCE

This application is a continuation of U.S. patent application Ser. No. 15/211,557, entitled “CELL CULTURE, TRANSPORT AND INVESTIGATION” filed Jul. 15, 2016 which claims priority to U.K. Patent Application No. 1512600.6, filed on Jul. 17, 2015, which applications are incorporated herein by reference.

### BACKGROUND

Cell cultures are of utility in medical research and life sciences in general. Traditional cell culture uses a simple container such as a petri dish or multi-well plate as a vessel for cell culture. However, it is recognized in the art that such a simple approach provides cells with a substantially different environment to that experienced by cells in vivo.

### SUMMARY

An aspect of the present disclosure provides a cell culture module. The cell culture module may comprise (a) an array comprising a plurality of chambers, each chamber of the plurality of chambers may be configured to house one or more cells and may be operatively coupled to a respective cell introduction port by a respective cell introduction passage; and (b) at least one perfusion channel may be fluidically coupled to a chamber of the plurality of chambers. In some embodiments, the array of chambers may be at least 3 chambers. In some embodiments, a volume of each chamber of the plurality of chambers may be at least about 4500 micrometers cubed (um³). In some embodiments, the plurality of chambers and a plurality of cell introduction passages may be formed at least partly in a monolithic matrix of the cell culture module. In some embodiments, the cell culture module may further comprise an upper region and a lower region of the cell culture module. The array may be formed in the lower region and a plurality of cell introduction ports may be formed in the upper region. In some embodiments, the at least one perfusion channel may be a plurality of perfusion channels being arranged in a stacked array. In some embodiments, the stacked array may be formed in the lower region, the upper region, or a combination thereof. In some embodiments, the at least one perfusion channel may be disposed substantially about an interface between the lower region and the upper region. In some embodiments, the at least one perfusion channel may be fluidically coupled to at least one associated perfusion port to form a conduit. The conduit may slope downwardly from the at least one associated perfusion port towards the chamber that may be fluidically coupled to the at least one perfusion channel. In some embodiments, an angle of the conduit relative to a horizontal axis of the array of chambers may be less than about 50 degrees. In some embodiments, a group of the plurality of perfusion channels of the stacked array may be disposed at a same horizontal height relative to the array. The group may be fluidically coupled to a common perfusion port. In some embodiments, the respective cell introduction passage may comprise a constriction. In some embodiments, the constriction may be oriented laterally relative to a horizontal axis of the array of chambers. In some embodiments, the constriction may be disposed adjacent to an entrance of a chamber of the plurality of chambers.

In some embodiments, the cell culture module may further comprise a moveable blocking element. The moveable blocking element may be configured to be moveable into a blocking configuration at the constriction to prevent a cell from exiting a chamber of the plurality of chambers. In some embodiments, the moveable blocking element may be configured to be moveable from the blocking configuration to an unblocking configuration. In some embodiments, the moveable blocking element may be a bead. In some embodiments, a movement of the bead to a blocking configuration or to an unblocking configuration may be directed by gravity, an applied magnetic field, an applied electric field, an applied fluid flow, a mechanical force, or any combination thereof. In some embodiments, the cell culture module may further comprise at least one bypass channel that fluidically couples (a) the chamber of the plurality of chambers to the respective cell introduction passage, (b) the chamber of the plurality of chambers to the at least one perfusion channel, or (c) a combination thereof. In some embodiments, when the moveable blocking element may be in the blocking configuration, the at least one bypass channel may permit (a) a fluid exchange between the chamber of the plurality of chambers and the respective cell introduction passage or the at least one perfusion channel, (b) an outgrowth of at least a portion of the cell from the chamber of the plurality of chambers into the respective cell introduction passage or the at least one perfusion channel, or (c) a combination thereof.

In some embodiments, an end of the respective cell introduction port may be disposed at an upper surface of the upper region of the cell culture module. In some embodiments, for each chamber of the plurality of chambers, a linear line of sight may form from an end of the respective cell introduction port disposed at the upper surface to a bottom internal surface of a respective chamber.

In some embodiments, the cell culture module may further comprise at least one electrode. In some embodiments, the at least one electrode may comprise a support portion and a head portion. In some embodiments, the support portion may operatively couple to an external circuitry. In some embodiments, the head portion may comprise a rounded shape of greater lateral width than the support portion. In some embodiments, the head portion may comprise a plurality of protrusions, a plurality of recesses, or a combination thereof In some embodiments, the at least one electrode may be formed on a substrate of the cell culture module. The monolithic matrix may surround at least a portion of the at least one electrode on the substrate. In some embodiments, the at least one electrode may comprise at least one protrusion, at least one recess, or a combination thereof. In some embodiments, the at least one electrode may comprise the at least one protrusion and the at least one protrusion may be a hemispherical protrusion. In some embodiments, the at least one electrode may comprise the at least one recess and the at least one recess may be a hemispherical recess. In some embodiments, the at least one electrode may comprise at least about 100 protrusions, at least about 100 recesses, or any combination thereof. In some embodiments, the at least one electrode may comprise a surface concentration of a plurality of protrusions of at least about 0.05 protrusions per micrometer squared (pro/um²), a surface concentration of a plurality of recesses of at least about 0.05 recesses per micrometer squared (rec/um²), or any combination thereof. In some embodiments, the at least one electrode may comprise a surface roughness from about 10 nanometers (nm) to about 100 nm. In some embodiments, a width of the at least one electrode may be from about 2 micrometers (um) to about 20 um. In some embodiments, a shape of the at least one electrode may be a mushroom shape.

In some embodiments, the cell culture module may further comprise one or more sensors. In some embodiments, the one or more sensors may comprise a temperature sensor, a pH sensor, a gas sensor, a glucose sensor, a level sensor, or any combination thereof. In some embodiments, the gas sensor may be an O₂ sensor, a CO₂ sensor, or a combination thereof. In some embodiments, the one or more sensors may comprise an optical sensor, an electrochemical sensor, an opto-electric sensor, a piezoelectric sensor, a biosensor, or any combination thereof. In some embodiments, each chamber of the plurality of chambers may comprise at least one sensor. In some embodiments, the cell culture module may further comprise a control system to direct the one or more sensors to make one or more measurements of a metric respective to the one or more sensors.

In some embodiments, the cell culture module may further comprise at least one light guide configured to conduct light from an external light source to a portion of the chamber of the plurality of chambers. In some embodiments, the at least one light guide may be a plurality of light guides. Each of the plurality of light guides may be configured to singularly correspondence to each of the plurality of chambers.

In some embodiments, a chamber of the plurality the chambers may be populated with at least two cells. In some embodiments, the cell culture module may further comprise a barrier arrangement configured to partition the at least one perfusion channel from an additional chamber, the additional chamber being for housing at least two cells. The barrier arrangement may comprise a plurality of pores to selectively permit a moiety to pass between the at least one perfusion channel and the additional chamber. In some embodiments, the barrier arrangement may be adapted to model a blood-brain barrier arrangement. In some embodiments, a pore diameter of the plurality of pores may be less than about 5 micrometers (um). In some embodiments, a pore diameter of the plurality of pores may be less than about 100 nanometers (nm). In some embodiments, a permeability of the barrier arrangement may be size-based or charge-based. In some embodiments, the moiety may be a gas, a chemokine, a cytokine, a small molecule, a protein, a nucleic acid, or any combination thereof.

Another aspect of the present disclosure provides a cell culture system comprising the cell culture module. The cell culture system may further comprise at least one temperature sensor configured to sense a temperature of at least a portion of the cell culture module. The cell culture system may further comprise at least one heating element configured to heat at least a portion of the cell culture module. The cell culture system may further comprise a fluid delivery system configured to deliver a fluid to the cell culture module. The cell culture system may further comprises a control system configured to control the heating element, the fluid delivery system, the at least one temperature sensor, or any combination thereof. The cell culture system may comprise any combination of the at least one temperature sensor, the at least one heating element, the fluid delivery system, or the control system. In some embodiments, the fluid delivery system may comprise a pump. In some embodiments, the pump may a positive displacement pump, an impulse pump, a velocity pump, a gravity pump, a steam pump, or a valveless pump. In some embodiments, the cell culture system may further comprise (a) a base unit configured to retain the fluid delivery system and the control system; and (b) a receiving region disposed on the cell culture module for releasably securing said cell culture module to the base unit and for operatively coupling the cell culture module to the fluid delivery system and the control system. In some embodiments, the control system may direct a supply of heat from the at least one heating element to the cell culture module such that a set temperature may be matched. In some embodiments, the set temperature may be input to the control system by a user.

Another aspect of the present disclosure provides a method of populating a cell culture module with the one or more cells. In some embodiments, the method may comprise inserting the one or more cells into each chamber of the plurality of chambers via the respective cell introduction passage. In some embodiments, the method may further comprise blocking the respective cell introduction passage with the movable blocking element.

Another aspect of the present disclosure provides a method of populating a cell culture module with the one or more cells. The method may comprise modifying at least a portion of a surface of the at least one electrode; and inserting the one or more cells into each chamber of the plurality of chambers via the respective cell introduction passage. In some embodiments, the one or more cell may contact at least a portion of the least one electrode. In some embodiments, the modifying may comprise adding a moiety to the portion of the surface. In some embodiments, the modifying may comprise increasing a surface roughness on the portion of the surface. In some embodiments, the modifying may comprise adding at least one protrusion, at least one recess, or a combination thereof adjacent to the portion of the surface. In some embodiments, the method may further comprise perfusing a fluid to a chamber of the plurality of chambers via the at least one perfusion channel.

Another aspect of the present disclosure provides a method of inspecting at least one cell of the cell culture module. In some embodiments, the cell may be located in a chamber of the plurality of chambers. The method may comprise visualizing the cell via the upper surface of the cell culture module employing a microscope.

Another aspect of the present disclosure provides a method comprising: applying a compound to the one or more cells contained in the cell culture module. In some embodiments, the method may further comprise adding the compound to the at least one perfusion channel and/or the respective cell introduction passage. In some embodiments, the method may further comprise measuring a response of the one or more cells to the compound. In some embodiments, the measuring may comprise employing a sensor to measure the response.

Another aspect of the present disclosure provides a method of manufacturing the cell culture module. The method may comprise 3D printing of at least a portion of the cell culture module.

In some embodiments, the at least one perfusion channel may be configured to introduce or to remove a fluid from the chamber. In some embodiments, the fluid may comprise a gas, a cell culture media, or a combination thereof. In some embodiments, the fluid may comprise a compound. In some embodiments, the one or more cells may comprise a neuron isolated from a subject. In some embodiments, the one or more cells may be obtained from a brain tissue, a spinal tissue, a cerebral spinal fluid, or any combination thereof. In some embodiments, the at least one perfusion channel may comprise an internal channel width of less than about 5 micrometers (um). In some embodiments, an internal channel width of the at least one perfusion channel may vary along its length. In some embodiments, an internal width of the respective cell introduction passage may vary along its length. In some embodiments, a width of the moveable blocking element may be equivalent to a smallest internal width along a length of the respective cell introduction passage. In some embodiments, the at least one perfusion channel may be fluidically coupled to each chamber of the plurality of chambers. In some embodiments, the at least one perfusion channel may comprise a plurality of fluidic branches. In some embodiments, an outer length and an outer width of the cell culture module may less than about 6 inches. In some embodiments, a length of the conduit may be more than about 1 millimeter (mm). In some embodiments, a volume of each chamber of the plurality of chambers may be from about 0.5 millimeters cubed (mm³) to about 90 mm³.

In some embodiments, the cell culture system may communicate a result obtained from the cell culture module via a communication media. In some embodiments, the communication media may be a personal computer, a cell phone, a tablet device, or a combination thereof.

In some embodiments, the cell culture module may comprise an array comprising a plurality of chambers, each chamber of the plurality of chambers may be configured to house one or more cells and may be operatively coupled to a respective cell introduction port by a respective cell introduction passage; and at least one perfusion channel may be fluidically coupled to a chamber of the plurality of chambers.

In some embodiments, the cell culture module may comprise an array comprising a plurality of chambers, each chamber of the plurality of chambers may be configured to house one or more cells and may be operatively coupled to its own cell introduction port by its own cell introduction passage; and at least one perfusion channel may be fluidically coupled to a chamber of the plurality of chambers.

The work in the art to date shows that there is interest in the development of complex systems in order to more closely mimic in vitro the behavior of cells in vivo. This allows, for example, the testing of candidate drug compounds on the cells, in order to assess efficacy and toxicity (for example) in an improved manner compared with simple testing on isolated cells in multiwell plates.

The most appropriate platform for drug development, biomarker research or understanding of human pathologies is the human system or the closest approximation thereof. To develop this closest approximation researchers are faced with significant challenges which often impact on productivity, increases cost and may produce inferior results due to the interdisciplinary nature of resources which may be required. These difficulties include but not limited to tissue sourcing and uniform differentiation for stem cell lines, regulatory issues, steep learning curves, labor, capital, material and time costs, data extraction and interpretation and so on.

The present disclosure has been devised in order to address at least one of the above problems. Preferably, the present disclosure reduces, ameliorates, avoids or overcomes at least one of the above problems.

Accordingly, in a first preferred aspect, the present disclosure provides a cell culture module configurable to be oriented to have an upper region and a lower region, the module comprising: an array of chambers in said lower region, each chamber being for housing one or more cells; for each chamber, a respective cell introduction passage and a respective cell introduction port, said cell introduction port being located in said upper region and being linked to said chamber via said cell introduction passage; at least one perfusion channel for conduction of cell culture media to and away from cells in said chambers, wherein at least said chambers and said cell introduction passages are formed at least partly in a monolithic matrix.

In a second preferred aspect, the present disclosure provides a cell culture system comprising a cell culture module according to the first aspect, the system further comprising: at least one temperature sensor, adapted to sense the temperature of at least part of the cell culture module; at least one heating element, adapted to heat at least part of the cell culture module; fluid delivery system (such as a system that delivers a cell media), adapted to deliver media to the cell culture module; and control system, adapted to control the heating element and/or the fluid delivery system. A fluid delivery system may include delivery of a cell culture media. A fluid delivery system may include delivery of a gas, such as oxygen or carbon dioxide. A fluid delivery system may include delivery of a reagent, such as an antibody, a protein, an enzyme, a small molecule, a nucleic acid, a cytokine, a chemokine, aptamer, or any combination thereof.

In a third preferred aspect, the present disclosure provides a method of populating a cell culture module according to the first aspect, including the steps of: inserting at least one cell into at least one chamber of the cell culture module via the cell introduction passage; blocking the cell introduction passage in order to retain the cell in the chamber; and perfusing cell culture media to the cell via the perfusion channel.

In a fourth preferred aspect, the present disclosure provides a method of testing a compound including the steps: providing a cell culture module according to the first aspect populated with cells; conducting said compound to the cells via the perfusion channel and/or the cell introduction passage; and assessing the response of at least one of the cells to the compound.

In a fifth preferred aspect, the present disclosure provides a method of manufacturing a cell culture module according to the first aspect including the step of 3D printing of the monolithic matrix in order to form the chambers and the cell introduction passages.

In a sixth preferred aspect, the present disclosure provides a method of inspecting at least one cell, the cell being located in a chamber of a module according to the first aspect, including carrying out optical microscopy on the chamber via the upper surface of the module.

The first, second, third, fourth, fifth and/or sixth aspect of the disclosure may have any one or, to the extent that they are compatible, any combination of the following optional features.

Preferably, the module further comprises a plurality of said perfusion channels. This allows cell culture media to perfuse through the module towards and away from the chambers. The perfusion channels may be arranged in a stacked array from the lower region to the upper region of the module. This allows at least the channels at or near the lower region of the module to remain filled with media, to ensure coverage of the cells in the chambers. For example, for ease of design and manufacture, when the module is oriented to have an upper region and a lower region, at least some of the perfusion channels may extend substantially horizontally across the module. However, as explained below, the perfusion channels may not be formed in a regular and/or linear array, where for example the structure of the perfusion channels is based on physiological modelling.

Each perfusion channel may have at least one associated perfusion port, providing a conduit from the exterior of the module in fluid communication with the respective perfusion channel, wherein, in the direction along said conduit from the perfusion port to the perfusion channel, said conduit slopes downwardly. Each perfusion channel may have more than one associated perfusion ports, such as 2, 3, 4 or more associated perfusion ports. In the stacked array of perfusion channels, there may be provided groups of perfusion channels provided at the same horizontal level as each other, each group of perfusion channels having at least one of said associated perfusion ports in common. This establishes a staked array of perfusion ports (similar in appearance to a gill-type system, for example), in which the slope of the conduit assists in retaining cell culture media in the perfusion channels of the module. In this way, the module is provided with a fluid retention system. The angling of the stacked array of perfusion ports, in communication with perfusion channels of relatively small diameter, provides a series of stacked resistances to fluid outflow. Such resistance to fluid outflow is of particular importance when the module is switched over after transport to a base unit of the system.

The cell introduction passage may include a constriction, such as a constriction in at least one lateral dimension. A constriction may be a circumferential constriction. A cell introduction passage may include one or more constrictions. A cell introduction passage may include at least two constrictions. A cell introduction passage may include at least three constrictions. A constriction may comprise a narrowing of an inner width of the cell introduction passage. A constriction may be oriented laterally relative to a horizontal axis of the array of chambers. A constriction may be disposed substantially adjacent to an entrance of a chamber. A constriction may be disposed at an interface between a chamber and a cell introduction passage, at which interface the chamber and cell introduction passage are fluidically coupled. This is useful in particular where the module further comprises a moveable blocking element. This element may be adapted to be moveable into a blocking configuration at the constriction in order to restrain a cell in the chamber from leaving the chamber. Additionally, the moveable blocking element may be adapted to be moveable from the blocking configuration to an unblocking configuration. For example, the moveable blocking element may be a bead which is capable of movement under the influence of an applied magnetic field and/or electric field.

Movement of a moveable blocking element may be controlled by a control system or a user or a combination thereof. Movement of the moveable blocking element may be directed from a blocking position to an unblocking position or vice versa by employing a magnetic field, an electric field, a gravitational force, a circumferentially restricting force, a lateral or vertical force, or any combination thereof.

A moveable blocking element may be a bead. A moveable blocking element may be a valve. A moveable blocking element may be a planar sheet, such as a slidable planar sheet. A moveable blocking element may be a constriction band that is capable of constricting and expanding to reduce and expand an inner diameter of a chamber opening, a channel, or a cell introduction passage. A moveable blocking element may be an array of posts.

Preferably, there is provided at least one bypass channel, permitting fluid flow and/or outgrowth of the cell from the chamber to the cell introduction passage or the perfusion channel, when the moveable blocking element is in the blocking configuration. The bypass channel preferably is dimensioned in order to prevent cells in the chamber from passing along the bypass channel.

An internal channel width of a bypass channel may be less than about 20 micrometers (um). An internal channel width of a bypass channel may be less than about 10 um. An internal channel width of a bypass channel may be less than about 5 um. An internal channel width of a bypass channel may be less than about 1 um. An internal channel width of a bypass channel may be less than about 0.5 um. An internal channel width of a bypass channel may be less than about 0.1 um. An internal channel width of a bypass channel may be less than about 0.05 um. An internal channel width of a bypass channel may be less than about 0.01 um.

An internal channel width of a bypass channel may vary along its length. An internal channel width of a bypass channel may be substantially the same along its length.

The cell introduction port is typically provided at an upper surface of the module, in the upper region of the module, the cell introduction passage sloping downwardly to the chamber. This slope assists in the introduction of cells into the chamber, and their retention in the chamber.

Preferably, for at least one chamber, a vertical line of sight exists from the upper surface of the module to the chamber without intersection with said cell introduction passage. This permits microscopic investigation of the cells in the chamber.

Preferably, the chamber includes at least one electrode, formed for interaction with the cell. The electrode may have a support portion and a head portion, the support portion being connected or connectable to external circuitry, the head portion having a rounded shape of greater lateral width than the support portion. The head portion of the electrode may include additional protrusions and/or depressions formed at the rounded shape, thereby providing additional available surface area compared with the rounded shape alone.

Preferably, the electrodes are formed on a substrate, the monolithic matrix being formed on the substrate in order to enclose the electrodes in the chambers. The module may further comprise at least one light guide, adapted to conduct light from an external light source to the chamber. An array of light guides may be provided, corresponding to at least part of the array of chambers.

In a preferred embodiment, the chambers are populated with one or more cells.

The walls of the cell introduction passage may deliberately be formed with roughness, steps or barbs, or otherwise be formed jagged). This may provide directionality such that cells inserted with some pressure may find it difficult to migrate out of the chamber along the passage. Additionally or alternatively, the walls of the cell introduction passage may be chemically functionalized, for example to prevent cells from growing along the cell introduction passage.

There may additionally be provided a barrier arrangement separating the perfusion channels linked with the chambers from at least one secondary chamber. The secondary chamber may be for housing one or more cells, preferably different from cells in said chambers. The barrier arrangement preferably includes an array of pores to allow selective communication between the perfusion channels and the secondary chamber.

It is preferred that the barrier arrangement is adapted to model a blood-brain barrier.

In the system, preferably there is provided a base unit retaining said fluid delivery system and said control system and having a cell culture module receiving region for releasably retaining said cell culture module and communicating said cell culture module to said fluid delivery system and said control system.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIGS. 1-17 show schematic views of a cell culture module according to an embodiment of the invention, in which different features of the module are indicated in the different drawings.

FIG. 18 shows a schematic transverse cross sectional view of a cell culture module according to an embodiment of the invention.

FIG. 19 shows a schematic internal perspective view of part of a cell culture module according to an embodiment of the invention.

FIG. 20 shows a schematic longitudinal cross sectional view of a cell culture module according to an embodiment of the invention.

FIG. 21 shows a schematic longitudinal cross sectional view of a cell culture module according to another embodiment of the invention.

FIGS. 22A and 22B show schematic cross sectional views of electrode structures for use with an embodiment of the invention.

FIGS. 23A and 23B show schematic views of electrode structures for use with an embodiment of the invention.

FIGS. 24A and 24B show enlarged schematic views of chambers for housing cells, for use in embodiments of the invention.

FIG. 25 shows a schematic cross sectional view of a chamber, lock-in bead and outgrowth/perfusion passages, for use with an embodiment of the invention.

FIGS. 26-30 illustrate steps in the manufacture of electrodes on a substrate, for use in an embodiment of the invention.

FIG. 31 shows a computer control system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “about” may be the referenced numeric indication plus or minus 15% of that referenced numeric indication.

The term “cell” as used herein, generally refers to one or more cells. A cell may be obtained or isolated from a subject. A subject may be an animal such as a human, a mouse, a rat, a pig, a dog, a rabbit, a sheep, a horse, a chicken or other. A cell may be a neuron. A neuron may be a central neuron, a peripheral neuron, a sensory neuron, an interneuron, a motor neuron, a multipolar neuron, a bipolar neuron, or a pseudo-unipolar neuron. A cell may be a neuron supporting cell, such as a Schwann cell. A cell may be one of the cells of a blood-brain barrier system. A cell may be a cell line, such as a neuronal cell line. A cell may be a primary cell, such as cells obtained from a brain of a subject. A cell may be a population of cells that may be isolated from a subject, such as a tissue biopsy, a cytology specimen, a blood sample, a fine needle aspirate (FNA) sample, or any combination thereof. A cell may be obtained from a bodily fluid such as urine, milk, sweat, lymph, blood, sputum, amniotic fluid, aqueous humour, vitreous humour, bile, cerebrospinal fluid, chyle, chyme, exudates, endolymph, perilymph, gastric acid, mucus, pericardial fluid, peritoneal fluid, pleural fluid, pus, rheum, saliva, sebum, serous fluid, smegma, sputum, tears, vomit, or other bodily fluid. A cell may comprise cancerous cells, non-cancerous cells, tumor cells, non-tumor cells, healthy cells, or any combination thereof.

The term “tissue” as used herein, generally refers to any tissue sample. A tissue may be a sample suspected or confirmed of having a disease or condition. A tissue may be a sample that is genetically modified. A tissue may be a sample that is healthy, benign, or otherwise free of a disease. A tissue may be a sample removed from a subject, such as a tissue biopsy, a tissue resection, an aspirate (such as a fine needle aspirate), a tissue washing, a cytology specimen, a bodily fluid, or any combination thereof. A tissue may comprise cancerous cells, tumor cells, non-cancerous cells, or a combination thereof. A tissue may comprise neurons. A tissue may comprise brain tissue, spinal tissue, or a combination thereof. A tissue may comprise cells representative of a blood-brain barrier. A tissue may comprise a breast tissue, bladder tissue, kidney tissue, liver tissue, colon tissue, thyroid tissue, cervical tissue, prostate tissue, lung tissue, heart tissue, muscle tissue, pancreas tissue, anal tissue, bile duct tissue, a bone tissue, uterine tissue, ovarian tissue, endometrial tissue, vaginal tissue, vulvar tissue, stomach tissue, ocular tissue, nasal tissue, sinus tissue, penile tissue, salivary gland tissue, gut tissue, gallbladder tissue, gastrointestinal tissue, bladder tissue, brain tissue, spinal tissue, a blood sample, or any combination thereof.

The term “compound” as used herein, generally refers to a composition that may produce an altered cellular response. A compound may be a drug or a pharmaceutical composition or salt thereof. A compound may be a protein, a peptide, a nucleic acid, an antibody, an aptamer, a small molecule. A compound may be a cell or a cellular fragment. A compound may be a tissue or tissue fragment. A compound may be a naturally-derived composition or a synthetic composition.

The term “surface roughness” as used herein, generally refers to surface texture or to an amplitude and/or a frequency of deviations on a surface. The deviations may be protrusions and/or recesses. The deviations may form a regular pattern or may be random.

The preferred embodiments of the present disclosure provide a cell culture module which contains, or is intended to contain, living cells. These cells may be from any suitable origin. For example, they may be simulated, synthetic or natural. The module may be intended to provide the cells with life support, capable of maintaining a suitable environment for these cells, including temperature control and delivering nutrients and other materials to the cells. As such, the module may be combined with other systems for the transportation of living cells, without interruption to normal physiological functions. The preferred embodiments of the disclosure provide electrodes for interaction with the cells, in order to monitor electrogenic signals. Preferred embodiments of the disclosure also allow the delivery of drugs or compounds to the cells and monitoring the cell response. Preferred embodiments of the disclosure may also support imaging modalities to monitor the cell response or function.

In some embodiments, the disclosure provides a fully autonomous, physiologically realistic multi organ on chip device. Such a device may be capable of supporting electrogenic cells with advanced cellular addressing, cellular membrane or barrier modelling and improved signal to noise ratio for electrogenic cells.

In general, the cell culture module of the preferred embodiment of the present disclosure provides a structure (which can be considered to be a substrate (in x, y, z coordinates)) in which the cells can be housed, the same structure providing an arrangement for perfusion of the cells with nutrients, growth media, growth factors, compounds, etc.

The cell culture module is intended to form part of a base unit, providing a gas delivery system which drives the perfusion of cell culture media through the cell culture module. Additionally, a cell culture system includes heating elements, for maintaining life support in the cell culture module. Various sensors are also included to monitor the temperature, pH, gas species, particle analysis, etc., in closed loop.

In addition to the foregoing, other sensors can be provided, e.g. adapted to sense the presence of specific proteins or ionic molecules. Such sensors and detectors can interface with the cell culture module in a secondary manner to provide analytic read outs for genomics, proteomics, western blot assays and other lab-on-chip devices.

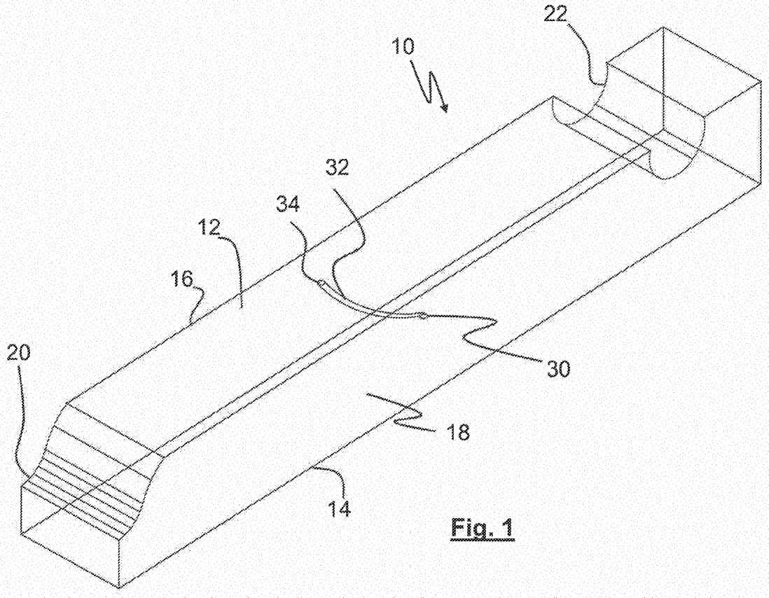

Turning now to FIGS. 1-17, these show schematic perspective views of a cell culture module according to an embodiment of the disclosure. Each drawing shows a different feature, or combination of features, in order to provide an understanding of the structure of this relatively complex module. In these drawings, the same features are indicated with the same reference numbers, and corresponding features between different drawings may not be discussed in detail for each drawing, it being understood that repeated explanation of the same features is unnecessary.

In FIG. 1, cell culture module is generally indicated by reference number 10. This has a generally elongate prismatic shape with an upper region bounded by upper surface 12 and a lower region bounded by lower surface 14. Upright side surfaces 16, 18 are also provided. Front region 20 is provided with a profile surface, for integration of the module into the cell culture system described later. Additionally, a cut-out 22 is formed towards the rear part of the upper region of the module. The front and rear shapes of the module provide purchase for a hooking system to be able to pick up the module and place it in a base unit (not shown).

The module may be manufactured by 3D printing, or similar microfabrication technology. This is described in more detail later. Such fabrication technologies are of interest in view of the need in the present case to be able to form complex interconnected passages and spaces for the location of cells, the perfusion of cell culture media and, where needed, cell outgrowth channels. These are described in more detail later.

In FIG. 1, chamber 30 is shown, located in the lower region of the module. Chamber 30 is intended to house one or more cells (not shown). Chamber 30 has a respective cell introduction passage 32, leading from cell introduction port 34 at upper surface 12 of the module. As can be seen in FIG. 1, the cell introduction passage slopes and curves generally downwardly from the upper region to the chamber in the lower region. This permits one or more cells to be introduced into chamber 30 via port 34 and passage 32, providing certainty as to the number and type of cells contained in chamber 30.

An outer length of the cell culture module may be less than about 12 inches. An outer length of the cell culture module may be less than about 11 inches. An outer length of the cell culture module may be less than about 10 inches. An outer length of the cell culture module may be less than about 9 inches. An outer length of the cell culture module may be less than about 8 inches. An outer length of the cell culture module may be less than about 7 inches. An outer length of the cell culture module may be less than about 6 inches. An outer length of the cell culture module may be less than about 5 inches. An outer length of the cell culture module may be less than about 4 inches. An outer length of the cell culture module may be less than about 3 inches. An outer length of the cell culture module may be less than about 2 inches. An outer length of the cell culture module may be less than about 1 inch. An outer length of the cell culture module may be less than about 0.5 inch.

An outer width of the cell culture module may be less than about 12 inches. An outer width of the cell culture module may be less than about 11 inches. An outer width of the cell culture module may be less than about 10 inches. An outer width of the cell culture module may be less than about 9 inches. An outer width of the cell culture module may be less than about 8 inches. An outer width of the cell culture module may be less than about 7 inches. An outer width of the cell culture module may be less than about 6 inches. An outer width of the cell culture module may be less than about 5 inches. An outer width of the cell culture module may be less than about 4 inches. An outer width of the cell culture module may be less than about 3 inches. An outer width of the cell culture module may be less than about 2 inches. An outer width of the cell culture module may be less than about 1 inch. An outer width of the cell culture module may be less than about 0.5 inch.

A chamber volume may be at least about 4 micrometers cubed (um³). A chamber volume may be less than about 4 micrometers cubed (um³). A chamber volume may be at least about 10 um³. A chamber volume may be less than about 10 um³. A chamber volume may be at least about 50 um³. A chamber volume may be less than about 50 um³. A chamber volume may be at least about 100 um³. A chamber volume may be less than about 100 um³. A chamber volume may be at least about 250 um³. A chamber volume may be less than about 250 um³. A chamber volume may be at least about 500 um³. A chamber volume may be less than about 500 um³. A chamber volume may be at least about 1000 um³. A chamber volume may be less than about 1000 um³. A chamber volume may be at least about 2000 um³. A chamber volume may be less than about 2000 um³. A chamber volume may be at least about 3000 um³. A chamber volume may be less than about 3000 um³. A chamber volume may be at least about 4000 um³. A chamber volume may be less than about 4000 um³. A chamber volume may be at least about 5000 um³. A chamber volume may be less than about 5000 um³. A chamber volume may be less than about 10,000 um³. A chamber volume may be at least about 10,000 um³. A chamber volume may be less than about 14,000 um³. A chamber volume may be at least about 14,000 um³. A chamber volume may be less than about 15,000 um³. A chamber volume may be at least about 15,000 um³. A chamber volume may be less than about 20,000 um³. A chamber volume may be at least about 20,000 um³. A chamber volume may be less than about 25,000 um³. A chamber volume may be at least about 25,000 um³. A chamber volume may be less than about 30,000 um³. A chamber volume may be at least about 30,000 um³. A chamber volume may be at least about 33,500 um³. A chamber volume may be less than about 33,000 um³.

A chamber volume may be at least about 0.01, 0.05, 0.1, 0.5, 1, 5, 10, 15, 20, 25, 30, 35, 40, 45, 50, 55, 60, 65, 70, 75, 80, 85, or 90 millimeters cubed (mm³). A chamber volume may be less than about 90 mm³. A chamber volume may be less than about 80 mm³. A chamber volume may be less than about 70 mm³. A chamber volume may be less than about 60 mm³. A chamber volume may be less than about 50 mm³. A chamber volume may be less than about 40 mm³. A chamber volume may be less than about 30 mm³.

A chamber volume may be from about 0.5 to about 90 mm³. A chamber volume may be from about 0.5 to about 80 mm³. A chamber volume may be from about 0.5 to about 70 mm³. A chamber volume may be from about 0.5 to about 60 mm³. A chamber volume may be from about 0.5 to about 50 mm³. A chamber volume may be from about 0.5 to about 40 mm³. A chamber volume may be from about 0.5 to about 30 mm³. A chamber volume may be from about 0.01 to about 10 mm³. A chamber volume may be from about 0.01 to about 1 mm³. A chamber volume may be from about 1 to about 30 mm³.

A chamber volume may be proportional to a volume of about a single cell. A chamber volume may be proportional to a volume of about 2 cells. A chamber volume may be proportional to a volume of about 5 cells. A chamber volume may be proportional to a volume of about 10 cells. A chamber volume may be proportional to a volume of about 15 cells. A chamber volume may be proportional to a volume of about 20 cells. A chamber volume may be proportional to a volume of about 25 cells. A chamber volume may be proportional to a volume of about 30 cells. A chamber volume may be proportional to a volume of about 40 cells. A chamber volume may be proportional to a volume of about 50 cells. A chamber volume may be proportional to a volume of about 60 cells. A chamber volume may be proportional to a volume of about 80 cells. A chamber volume may be proportional to a volume of about 100 cells. A chamber volume may be proportional to about 2, 3, 4, 5, 6, 7, 8, 9, 10, 15, 20, 25, 30, 35, 40, 45, 50, 55, 60, 65, 70, 75, 80, 100 cells or more.

A chamber volume may be proportional to a cell volume from about 1 cell to about 5 cells. A chamber volume may be proportional to a cell volume from about 1 cell to about 10 cells. A chamber volume may be proportional to a cell volume from about 1 cell to about 20 cells. A chamber volume may be proportional to a cell volume from about 1 cell to about 50 cells.

A chamber volume may accommodate 1 cell division of a single cell. A chamber volume may accommodate 2 cell divisions of a single cell. A chamber volume may accommodate 3 cell divisions of a single cell. A chamber volume may accommodate 4 cell divisions of a single cell. A chamber volume may accommodate 5 cell divisions of a single cell. A chamber volume may accommodate 6 cell divisions of a single cell. A chamber volume may accommodate 7 cell divisions of a single cell. A chamber volume may accommodate 8 cell divisions of a single cell.

A chamber volume may accommodate 1 cell division of two cells. A chamber volume may accommodate 2 cell divisions of two cells. A chamber volume may accommodate 3 cell divisions of two cells. A chamber volume may accommodate 4 cell divisions of two cells. A chamber volume may accommodate 5 cell divisions of two cells. A chamber volume may accommodate 6 cell divisions of two cells. A chamber volume may accommodate 7 cell divisions of two cells. A chamber volume may accommodate 8 cell divisions of two cells.

A chamber may comprise one or more cells. A chamber may comprise one or more beads, such as one or more magnetic beads. A chamber may comprise one or more cells, one or more beads, one or more bacterium, one or more yeast, one or more viruses, or any combination thereof.

A conduit slope may be defined as the angle of a conduit relative to an array of chambers in an lower region of a cell culture module. A conduit slope may be about 5, 10, 15, 20, 25, 30, 35, 40, 45, 50, 55, 60, 65, 70, 75, 80, or 85 degrees. A conduit slope may be about 20 degrees. A conduit slope may be about 25 degrees. A conduit slope may be about 30 degrees. A conduit slope may be about 35 degrees. A conduit slope may be about 40 degrees. A conduit slope may be about 45 degrees. A conduit slope may be about 50 degrees. A conduit slope may be about 55 degrees. A conduit slope may be about 60 degrees. A conduit slope may be about 65 degrees. A conduit slope may be about 70 degrees. A conduit slope may be about 75 degrees. A conduit slope may be about 80 degrees.

A conduit slope may be less than about 5, 10, 15, 20, 25, 30, 35, 40, 45, 50, 55, 60, 65, 70, 75, 80, or 85 degrees. A conduit slope may be less than about 20 degrees. A conduit slope may be less than about 25 degrees. A conduit slope may be less than about 30 degrees. A conduit slope may be less than about 35 degrees. A conduit slope may be less than about 40 degrees. A conduit slope may be less than about 45 degrees. A conduit slope may be less than about 50 degrees. A conduit slope may be less than about 55 degrees. A conduit slope may be less than about 60 degrees. A conduit slope may be less than about 65 degrees. A conduit slope may be less than about 70 degrees. A conduit slope may be less than about 75 degrees. A conduit slope may be less than about 80 degrees.

A conduit slope may be from about 5 to about 80 degrees. A conduit slope may be from about 5 to about 55 degrees. A conduit slope may be from about 30 to about 80 degrees. A conduit slope may be from about 30 to about 55 degrees. A conduit slope may be from about 55 to about 80 degrees. A conduit slope may be from about 20 to about 40 degrees. A conduit slope may be from about 40 to 60 degrees. A conduit slope may be from about 60 to about 80 degrees.

A length of a conduit may be more than about 0.01 millimeters (mm). A length of a conduit may be more than about 0.1 mm. A length of a conduit may be more than about 0.5 mm. A length of a conduit may be more than about 1 mm. A length of a conduit may be more than about 2 mm. A length of a conduit may be more than about 5 mm. A length of a conduit may be more than about 10 mm. A length of a conduit may be more than about 20 mm. A length of a conduit may be more than about 30 mm. A length of a conduit may be more than about 50 mm. A length of a conduit may be from about 1 mm to about 10 mm. A length of a conduit may be from about 0.5 mm to about 5 mm. A length of a conduit may be from about 0.1 to about 5 mm. A length of a conduit may be from about 0.1 mm to about 1 mm.

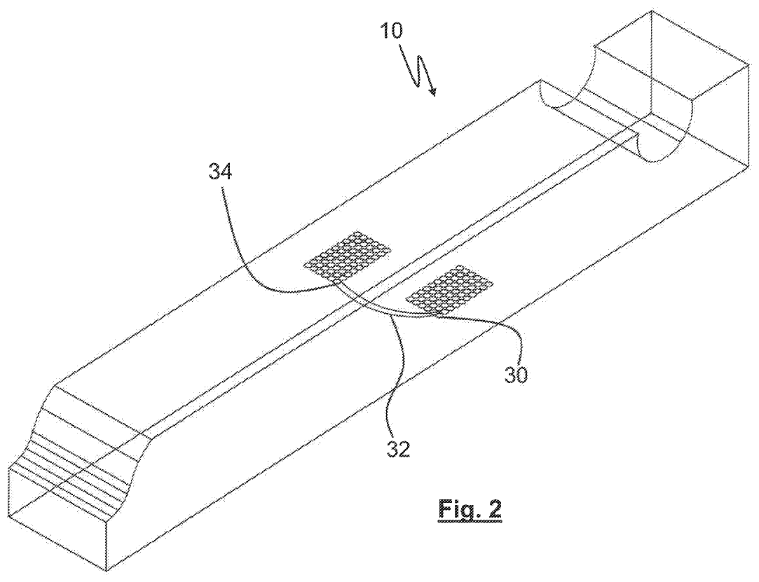

As shown in FIG. 2, chamber 30 is one of an array of chambers formed in the lower region of the module 10. In this embodiment, the array is a 10×6 array. Each chamber 30 has a respective cell introduction port 34, and a respective cell introduction passage 32, although only one cell introduction passage 32 is shown in FIG. 2, for clarity. Thus, it can be seen that introduction of a cell or cells into one cell introduction port 34, along the corresponding cell introduction passage 32, results in the cell or cells being located in the respective chamber, without risk of locating that cell or cells in a different chamber 30.

An array of chambers may be 2 or more chambers. An array of chambers may be 5 or more chambers. An array of chambers may be 10 or more chambers. An array of chambers may be 20 or more chambers. An array of chambers may be 50 or more chambers. An array of chambers may be 100 or more chambers. An array of chambers may be 200 or more chambers. An array of chambers may be 500 or more chambers. An array of chambers may be 3, 4, 5, 6, 7, 8, 9, 10, 15, 20, 25, 30, 35, 40, 45, 50, 100, 150, 200, 250, 300, 350, 400, 450, 500 or more chambers.

An array of chambers may be less than 500 chambers. An array of chambers may be less than 200 chambers. An array of chambers may be less than 100 chambers. An array of chambers may be less than 50 chambers. An array of chambers may be less than 20 chambers. An array of chambers may be less than 10 chambers. An array of chambers may be less than 5 chambers.

An array of chambers may be from 2 to 500 chambers. An array of chambers may be from 2 to 400 chambers. An array of chambers may be from 2 to 300 chambers. An array of chambers may be from 2 to 200 chambers. An array of chambers may be from 2 to 100 chambers. An array of chambers may be from 2 to 50 chambers. An array of chambers may be from 2 to 20 chambers. An array of chambers may be from 2 to 10 chambers. An array of chambers may be from 2 to 5 chambers.

A plurality of chambers may be 2 or more chambers. A plurality of chambers may be 5 or more chambers. An plurality of chambers may be 10 or more chambers. A plurality of chambers may be 20 or more chambers. A plurality of chambers may be 50 or more chambers. A plurality of chambers may be 100 or more chambers. A plurality of chambers may be 200 or more chambers. A plurality of chambers may be 500 or more chambers. A plurality of chambers may be 3, 4, 5, 6, 7, 8, 9, 10, 15, 20, 25, 30, 35, 40, 45, 50, 100, 150, 200, 250, 300, 350, 400, 450, 500 or more chambers.

A plurality of chambers may be less than 500 chambers. A plurality of chambers may be less than 200 chambers. A plurality of chambers may be less than 100 chambers. A plurality of chambers may be less than 50 chambers. A plurality of chambers may be less than 20 chambers. A plurality of chambers may be less than 10 chambers. A plurality of chambers may be less than 5 chambers.

A plurality of chambers may be from 2 to 500 chambers. A plurality of chambers may be from 2 to 400 chambers. A plurality of chambers may be from 2 to 300 chambers. A plurality of chambers may be from 2 to 200 chambers. A plurality of chambers may be from 2 to 100 chambers. A plurality of chambers may be from 2 to 50 chambers. A plurality of chambers may be from 2 to 20 chambers. A plurality of chambers may be from 2 to 10 chambers. A plurality of chambers may be from 2 to 5 chambers.

An array density may be about 0.2, 0.15, 0.1, 0.09, 0.08, 0.07, 0.06, 0.05, 0.04, 0.03, 0.02, 0.01, chambers per micrometer (chambers/um). An array density may be about 0.1 chambers/um or less. An array density may be about 0.15 chambers/um or less. An array density may be about 0.2 chambers/um or less. An array density may be about 0.25 chambers/um or less. An array density may be about 0.3 chambers/um or less. An array density may be about 0.35 chambers/um or less. An array density may be about 0.4 chambers/um or less. In some cases, outgrowth of a cell, such as a neuronal cell, from a chamber into a perfusion channel determines an array density.

A chamber shape may be a uniform shape, such as a sphere, a cube, a bulb-like shape, or a pear-like shape. A chamber shape may be a non-uniform shape or comprise irregularities. A chamber shape may mimic a biostructure or an in vivo tissue structure. A chamber shape may mimic a neuronal synapse. A chamber shape may mimic an axon terminal. A chamber shape may mimic a presynaptic cell.

A chamber may comprise a bead, a cell or a combination thereof. A chamber may comprise more than one bead. A bead may be attached to a cell and thereby introduced into a chamber when the cell is introduced into the chamber. A bead may be associated with a portion of a chamber, such as bound to an inner surface. A bead may be associated with a chamber, such as occupying a volume within the chamber. A bead may be configured to block a cell from exiting a chamber, such as a bead that is moveable to and from an exit port of the chamber. A bead may bind to a cell, a portion of the chamber, or a combination thereof. A bead may be a unique bead, such as a bead comprising a unique identifier. A bead may be a known bead, such as a bead comprising a known identifier. A bead may identify a cell. A bead may identify a chamber.

One or more chambers of a cell culture module may be coated with a substrate. A portion of a chamber may be coated with a substrate. The substrate may facilitate cell attachment. The substrate may reduce or prevent cell attachment. For example, portions of a chamber than are near an opening or cell introduction passage may be coated with a substrate that prevents cell attachment to prevent a cell from migrating out of the chamber. A substrate may facilitate temperature maintenance of a chamber. For example, a substrate may comprise a conductive material. A substrate may facilitate optical viewing in a chamber. For example, a substrate may be transparent or semi-transparent or permit light to pass through without distortion. A channel, such as a cell introduction passage, may be coated with a substrate, such as a substrate that reduces or prevents cell attachment.

A chamber may be configured to comprise a substrate upon which a cell is cultures and that is configured to provide an electrical interaction between one or more external components of the cell culture module and the cell. A substrate may be configured to facilitate capturing an output from a cell and/or exporting the output to an external component of the cell culture module. For example, the substrate may comprise an electrode to capture an electrical signal from a cell. A substrate may be configured to transmit or to conduct a stimulus from an external component to the cell. For example, the substrate may be configured to maintain a temperature of a cell by providing by an external heating element to the surface substrate. A substrate may comprise one or more sensors. A substrate may comprise a surface roughness.

One or more biological samples may be added to one or more chambers of the cell culture system. A biological sample added to a chamber may be a single cell. A biological sample may be two cells. A biological sample may be 3, 4, 5, 10, 20, 50, 100, 200, 500 or more cells. A biological sample may be a tissue section, a biopsy, a tissue resection, a bodily fluid, or a tissue aspirate. A biological sample may be an organoid or 3D cell culture sample. A biological sample may comprise a homogeneous population of cells. A biological sample may comprise a heterogeneous population of cells. A biological sample may comprise a known population such as a single neuron. A biological sample may comprise an undefined population such as a tissue biopsy from a subject.

A biological sample added to a chamber may be cultured in the cell culture system for at least about 1 day and remain viable and retain its phenotypical characteristics. For example, an insulin-producing cell added to a chamber may remain viable and continue to produce insulin for at least about 1 day in the cell culture system. For example, a GABA-expressing neuron added to a chamber may remain viable and continue to express GABA for at least about 1 day in the cell culture system.

A biological sample may remain viable for at least about 1, 2, 3, 4, 5, 6, 7, 14, 28, 30, 60, 100, 150, 200, 250, 350, 365 days or more. A biological sample cultured in the cell culture system may remain viable for at least about 7 days. A biological sample cultured in the cell culture system may remain viable for at least about 14 days. A biological sample cultured in the cell culture system may remain viable for at least about 28 days. A biological sample cultured in the cell culture system may remain viable for at least 60 days. A biological sample cultured in the cell culture system may remain viable for at least 90 days. A biological sample cultured in the cell culture system may remain viable for at least 120 days.

A biological sample may retain its phenotypic characteristics for at least about 1, 2, 3, 4, 5, 6, 7, 14, 28, 30, 60, 100, 150, 200, 250, 350, 365 days or more. A biological sample cultured in the cell culture system may retain its phenotypic characteristics for at least about 7 days. A biological sample cultured in the cell culture system may retain its phenotypic characteristics for at least about 14 days. A biological sample cultured in the cell culture system may retain its phenotypic characteristics for at least about 28 days. A biological sample cultured in the cell culture system may retain its phenotypic characteristics for at least 60 days. A biological sample cultured in the cell culture system may retain its phenotypic characteristics for at least 90 days. A biological sample cultured in the cell culture system may retain its phenotypic characteristics for at least 120 days.

The primary cell introduction array, illustrated in FIG. 2, allows cells to be inserted into the module in defined way, so that the location or address is known and the cells are locked in that location. The cell may be assisted in reaching the chamber using a magnetic field, electric field, chemical coating, gravity or any suitable combination.

The monolith is preferably a transparent monolith. The manufacture of device can be accomplished by may be accomplished current lithographic technologies or by multiphoton lithography using light manipulated in 3D space. The light manipulation may be accomplished with standard scanners, holographic technologies or other spatial or temporal light modulating technologies, micro mirror array technologies or associated methods. The use of a transparent material for the monolith matrix is particularly advantageous because it allows the cells to be viewed from the top of side of the module using laser scanning technologies, to assess the health, response, etc. of the cells.

The specific structure of the cell introduction passage 32 will be described in more detail later. Clearly, it should have a diameter suitable to permit the introduction of a desired cell or cells. Depending on the nature of the cells intended to be housed in chamber 30, the size of chamber 30, the diameter of cell introduction port 34 and the diameter of cell introduction passage 32 can be designed accordingly.

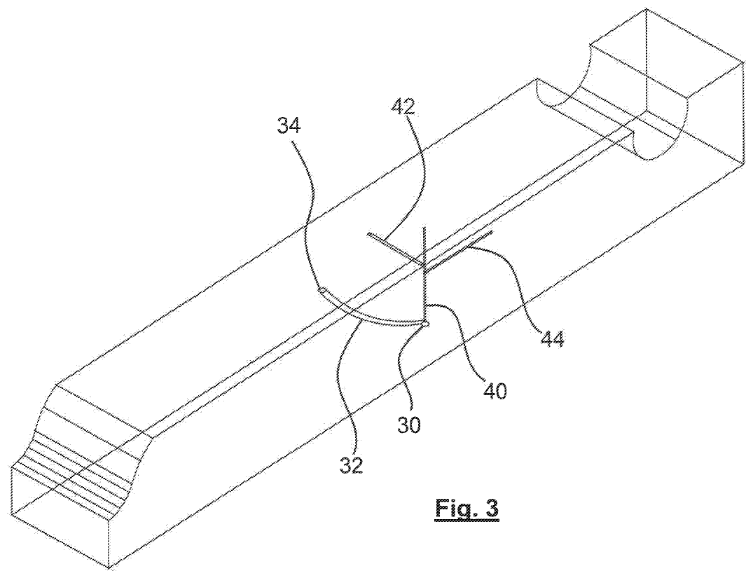

FIG. 3 schematically illustrates a set of perfusion channels in fluid communication with chamber 30. For the sake of ease of drawing, these perfusion channels are shown as straight and orthogonal to each other. However, the perfusion channels may be in any suitable form, and in particular may be based on physiologically-modelled shapes, as such, they may be tortuous rather than straight, and may extend in different directions to those illustrated.

An internal channel width of a perfusion channel may be about 0.01, 0.02, 0.03, 0.04, 0.05, 0.06, 0.07, 0.08, 0.09, 0.1, 0.2, 0.3, 0.4. 0.5, 0.6, 0.7, 0.8, 0.9, 1, 2, 3, 4, 5, 6, 7, 8, 10, 15, 20 micrometers (um). An internal channel width of a perfusion channel may be less than about 5 um. An internal channel width of a perfusion channel may be less than about 1 um. An internal channel width of a perfusion channel may be less than about 0.5 um. An internal channel width of a perfusion channel may be less than about 0.1 um. An internal channel width of a perfusion channel may be smaller than an outer width of the moveable blocking element. An internal channel width of a perfusion channel may be smaller than an internal width of a cell introduction passage.

A plurality of perfusion channels can be arranged in a stacked array. A stacked array can be a vertically stacked array, a horizontally stacked array, or a combination thereof.

In FIG. 3, there is shown a vertical perfusion channel 40, lateral perfusion channel 42 and longitudinal perfusion channel 44. Vertical perfusion channel 40 is in direct fluid communication with chamber 30. Lateral perfusion channel 42 is in fluid communication with the vertical perfusion channel 40. Similarly, longitudinal perfusion channel 44 is in fluid communication with vertical perfusion channel 40. As will be understood, the perfusion channels may have different arrangements.

The perfusion channels have dimensions which are small enough to prevent a cell present in chamber 30 from moving along the perfusion channel. For example, the diameter of a perfusion channel in direct fluid communication with chamber 30 is preferably at most 50% of the cell diameter of cells intended to be located in chamber 30.

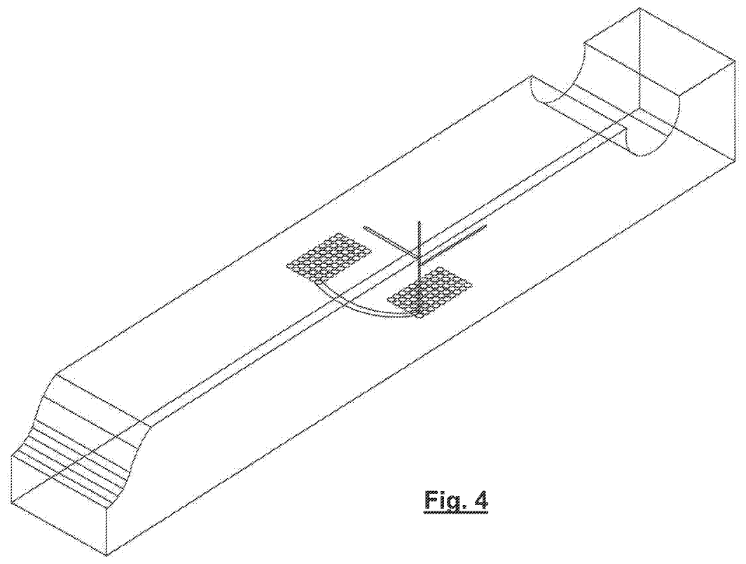

FIG. 4 shows a view corresponding to FIG. 3 in combination with FIG. 2.

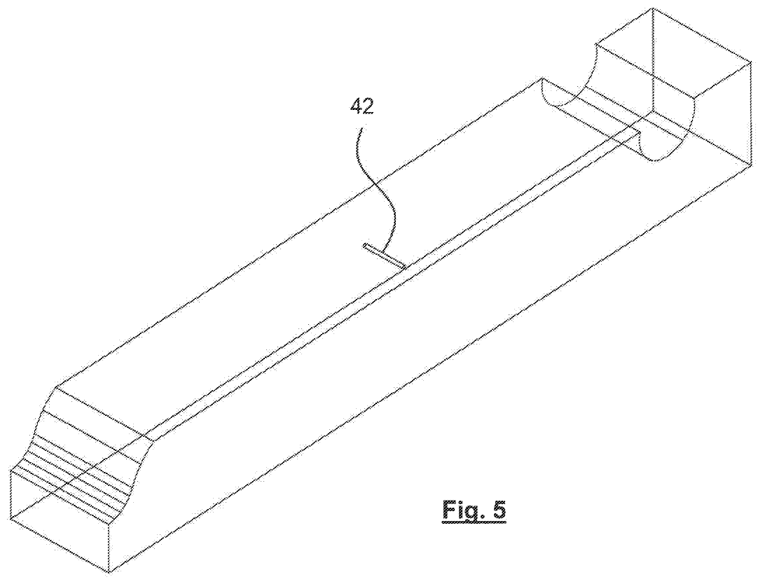

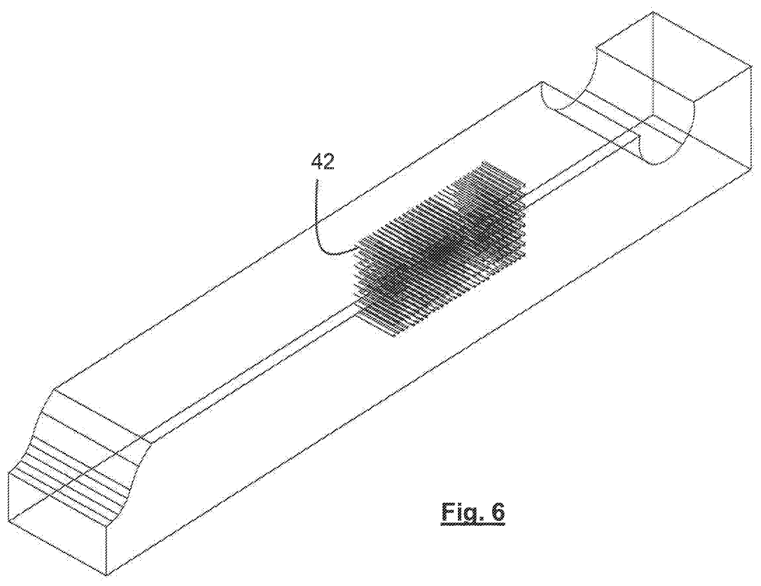

FIG. 5 shows the module with one transverse perfusion channel 42. FIG. 6 shows the module with an array of transverse perfusion channels 42. As will be understood, there is no view shown in which there is an array of transverse, longitudinal and vertical perfusion channels, because the drawing would become too dense to be understandable.

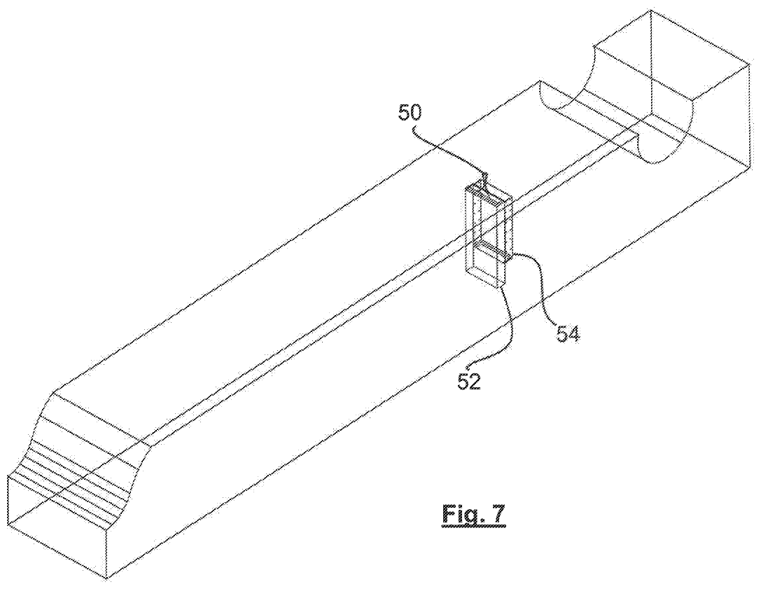

In FIG. 7, the module is shown having a secondary cell introduction port 50 leading to secondary chamber 52, shown in the drawing as having a generally cuboid shape, although the present disclosure is not necessarily limited to this nor is it necessarily limited to the orientation shown. Disposed adjacent secondary chamber 52, and to the rear of it, is secondary reservoir 54, intended to provide a reservoir for cell culture media.

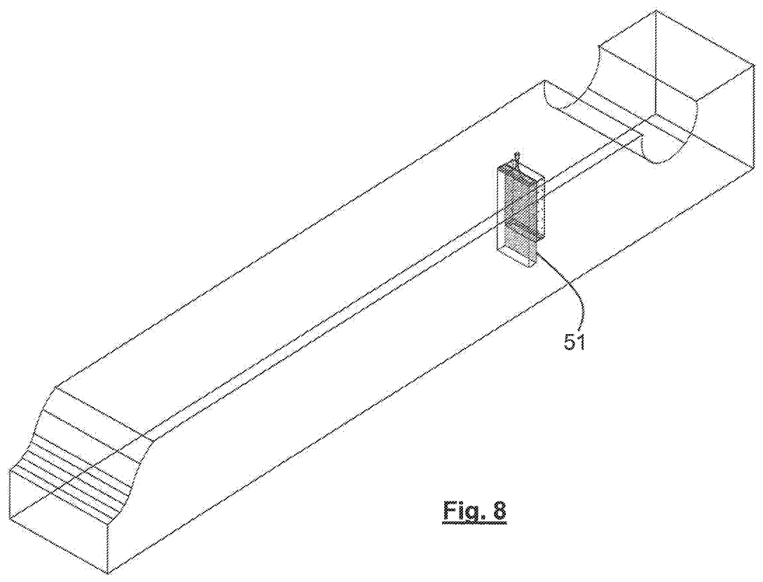

As shown in FIG. 8, there is provided a membrane structure 51 between the secondary chamber 52 and the secondary reservoir 54, membrane structure 51 having openings of size less than the size of cells contained in the secondary chamber, to permit fluid to flow between the secondary chamber 52 and the secondary reservoir 54,

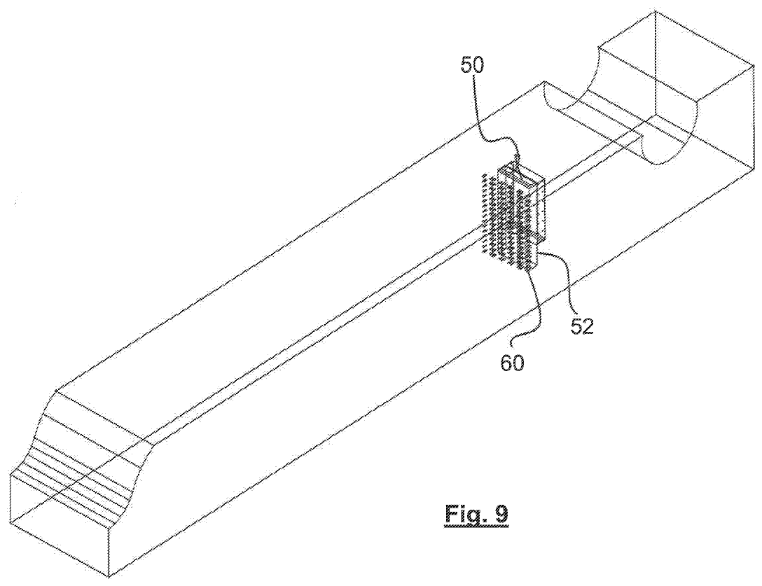

FIG. 9 shows a view corresponding to that shown in FIG. 7, but with barrier arrangement 60 formed at the forward side of secondary chamber 52. Barrier arrangement 60 consists of an array of pores opening into secondary chamber 52. In FIG. 9, the array of pores is shown as being a regular array, but this is not necessarily needed.

A barrier arrangement may comprise a plurality of pores. A pore diameter may be less than about 10, 9, 8, 7, 6, 5, 4, 3, 2, 1 micrometers (um). A pore diameter may be less than about 1, 0.5, 0.1, 0.05, 0.01 um. A pore diameter may be less than about 5 um. A pore diameter may be less than about 1 um. A pore diameter may be less than about 0.5 um. A pore diameter may be less than about 0.1 um. A permeability of the barrier arrangement may be (a) size-based, such as based on a pore size, (b) charge-based, such as based on an overall net charge, or (c) a combination thereof. A barrier arrangement may selectively permit a moiety to pass through a pore of the barrier arrangement. A moiety may comprise a gaseous molecule, a chemokine, a cytokine, a small molecule, a protein or fragment thereof, a nucleic acid, a cell or fragment thereof, or any combination thereof. A cell culture module may comprise one or more barrier arrangements. A cell culture module may comprise a barrier arrangement for each of the chambers of the plurality of chambers. A barrier arrangement may partition a perfusion channel from a chamber such as to selectively permit a moiety to pass between the perfusion channel and the chamber.

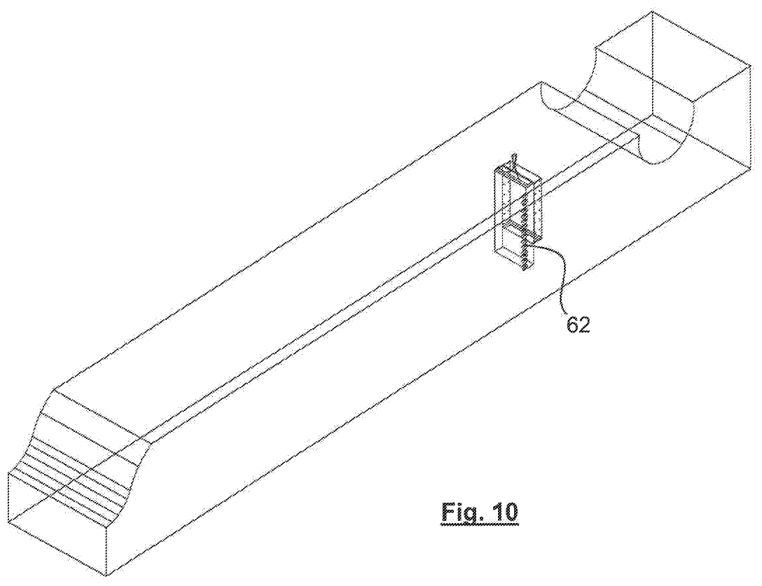

FIG. 10 shows a part of the barrier arrangement, being a series of pores 62.

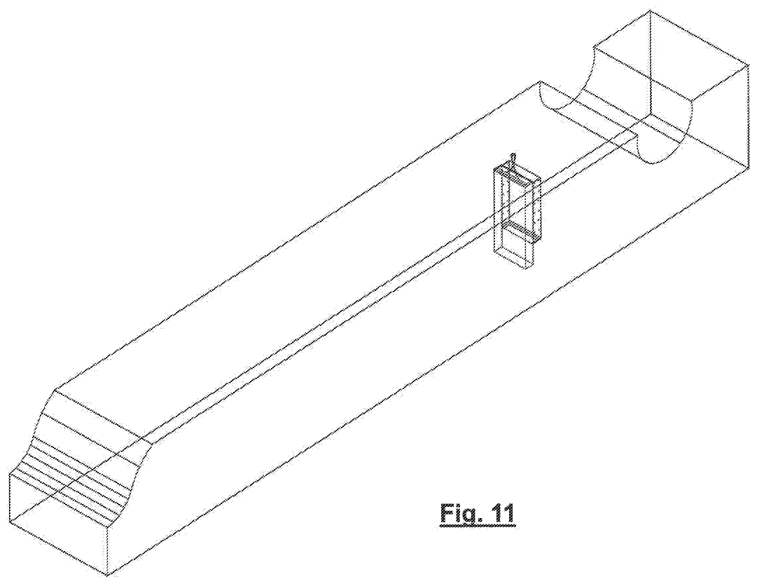

FIG. 11 shows a similar view to FIG. 7.

Secondary insertion port 50 provides access to the secondary chamber 52. As for the primary cell insertion array, this can be accessed by using an external system to address the required locations and introduce a cell of choice. It is also be possible to do same manually especially with organoids, slices, biopsy sample or whole brain organoids.

The primary cell insertion array allows the formation of a network of cells and the secondary chamber allows for the formation of monolayers or other configurations of choice.

There may be provided additional secondary cell insertion ports. The cells in the secondary chamber allow for flexibility of observing other cells in relation to the output of another group of cells. For example, where spinal or cortical neurons are used in the primary cell chambers, muscle cells may be used in the secondary chamber. This allows the user to observe the electrical signals or monitor the reaction via other methods, e.g., microscopy.

The secondary chamber allows for the functionalization of a wall of the chamber with secondary polymers. This wall may be the barrier arrangement.

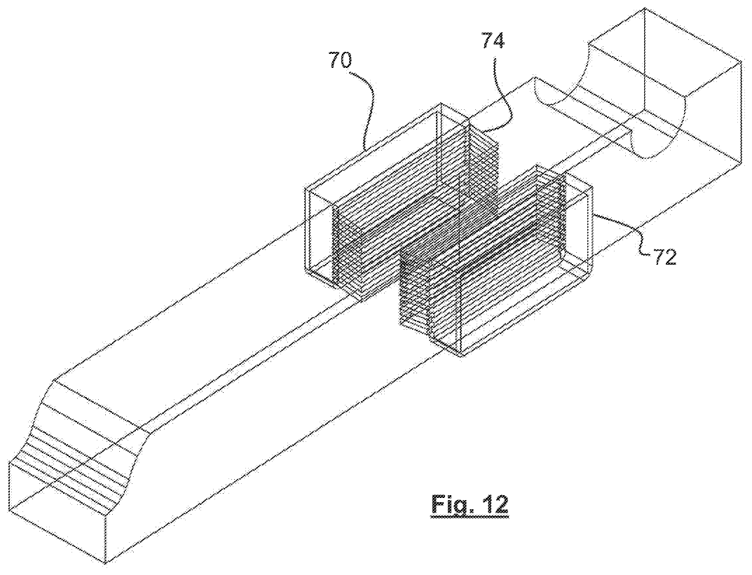

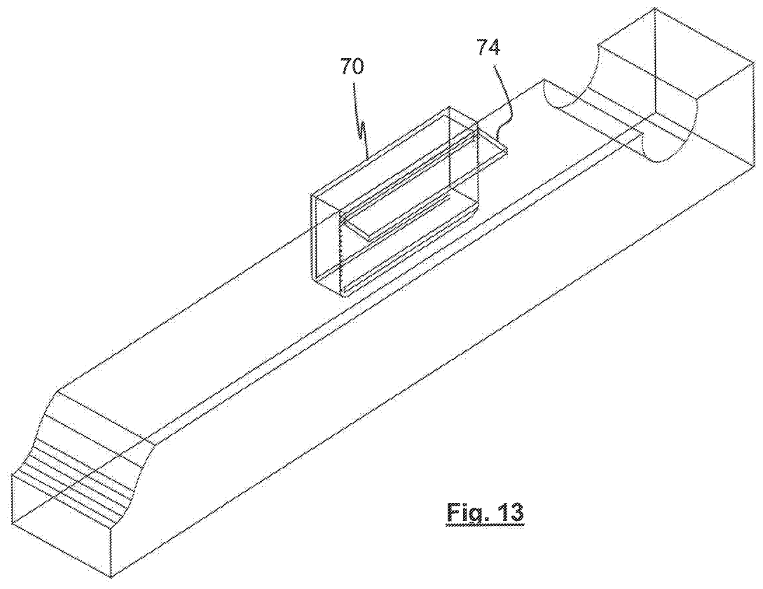

FIGS. 12 and 13 show more detail relating to the perfusion of cell culture media. In FIG. 12, side ports 70, 72 are provided at sides 16, 18 of the module, respectively. Side ports 70, 72 provide fluid communication with perfusion conduits 74. As shown in FIG. 13, perfusion conduit 74 takes the form of an elongate slot which slopes gently downwardly from perfusion port 70. This slope is intended to help to retain the cell culture media in the module against the force of gravity.

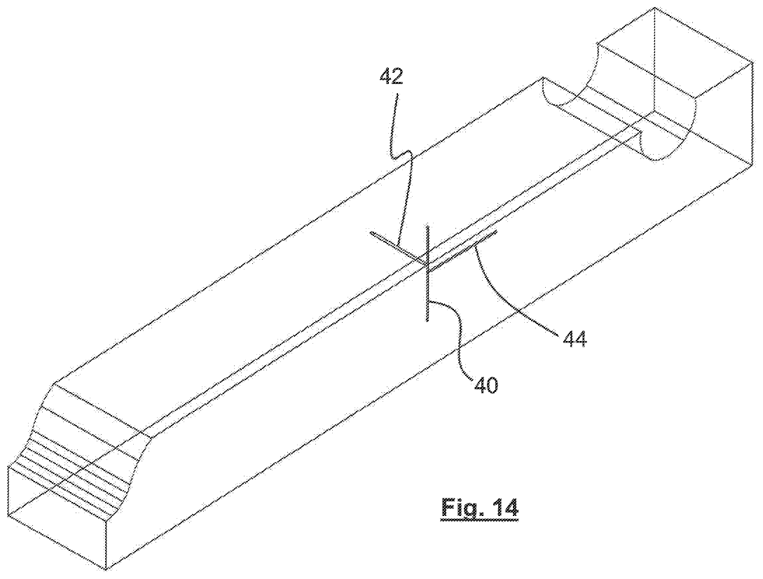

FIG. 14 shows the module with the perfusion channels 40, 42 and 44 already described.

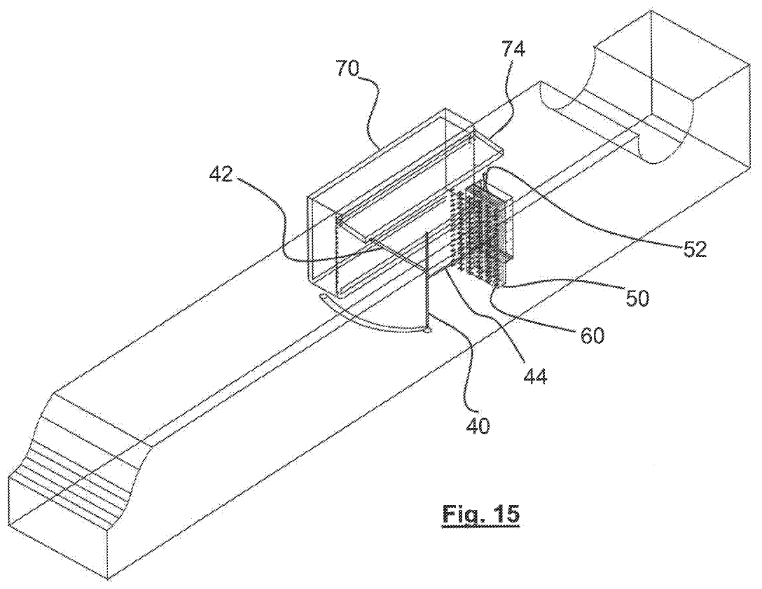

FIG. 15 shows the fluid communication between perfusion conduit 74 and lateral perfusion channel 42. In turn, lateral perfusion channel 42 is in fluid communication with vertical perfusion channel 40. In turn, vertical perfusion channel 40 is in fluid communication with longitudinal perfusion channel 44. In turn, longitudinal perfusion channel 44 leads towards barrier arrangement 60.

To support cell growth, the living cells should be perfused with growth media. Therefore the network of perfusion channels allows fluids to move round the system. The fluid entering and exiting the monolith is pretreated with gas, controlled for temperature, pH, chemical contents, dissolved proteins before being fed into the module. The media output is also analyzed post perfusion.

The arrangement of perfusion conduits shown in FIGS. 12 and 13 forms a horizontal gill system which helps to ensure that the cells are safely covered with cell culture media during transportation. The arrangement of perfusion channels between the gill systems provides some resistance to fluid flow, thereby additionally serving to retain and control fluid flow.

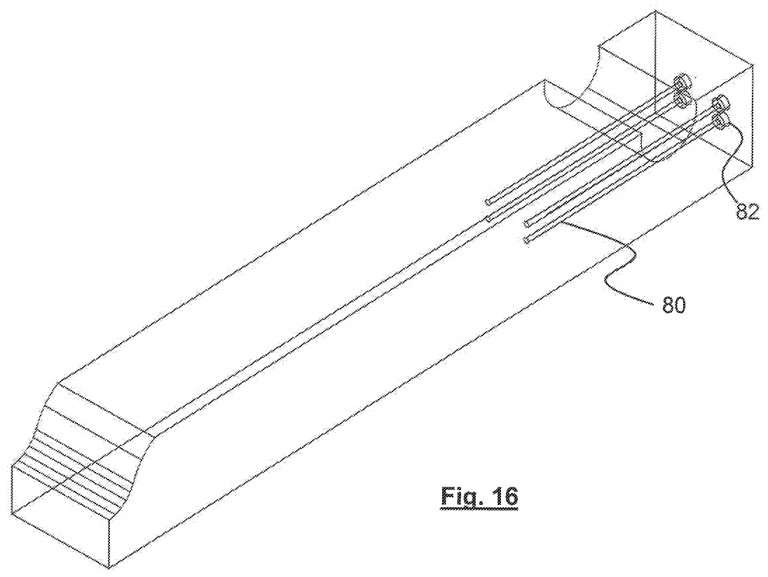

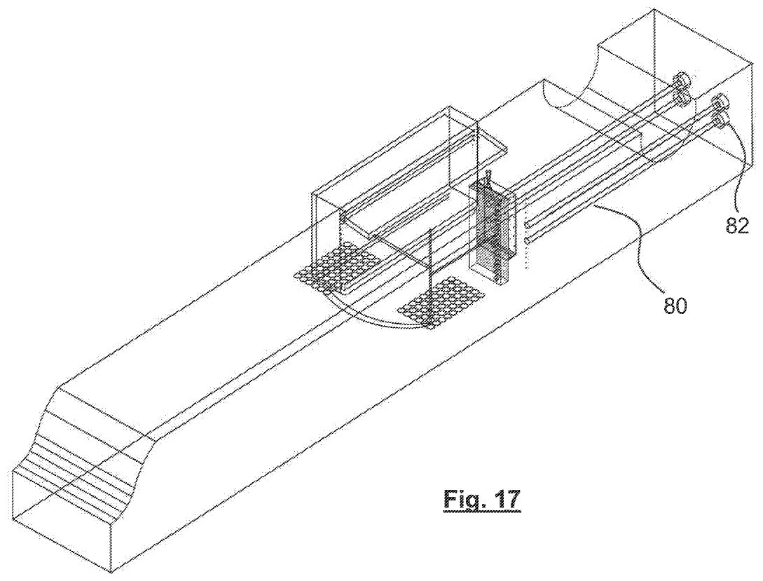

In FIG. 16, the module is shown with an array of light guides 80 extending in to the module from light ports 82 located at the rear end face of the module. Light guides 80, as shown in FIG. 17, extend longitudinally into the module to reach reservoir 54.

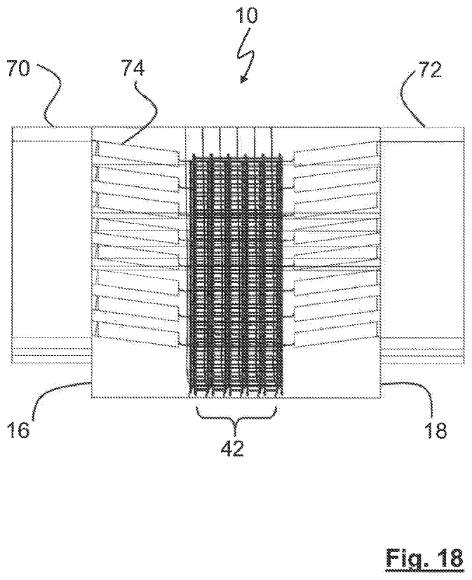

FIG. 18 shows a schematic transverse cross sectional view of the module 10. Perfusion ports 70, 72 are shown attached at the side surfaces 16, 18 of the module. Perfusion conduits 74 are here shown more clearly as sloping gently downwards towards the network of perfusion channels, indicated generally as 46.

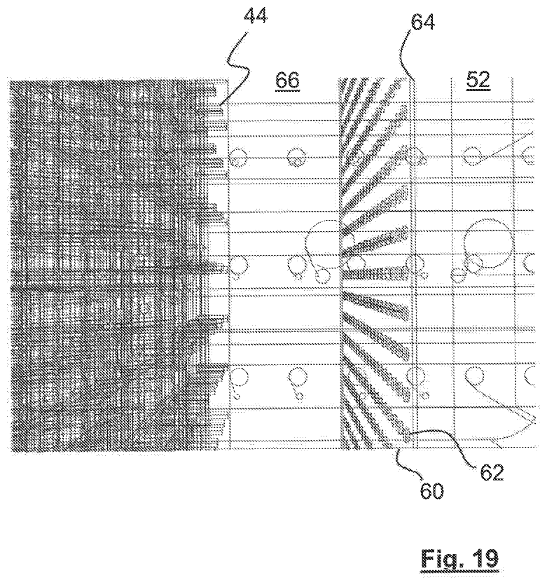

FIG. 19 shows a schematic perspective internal view of part of the module, intended to show the interaction between the perfusion channels and the barrier arrangement. The barrier arrangement 60 is shown as an array of cylindrical pores 62 extending longitudinally through a matrix wall 64 of the module. On the right hand side of barrier arrangement 60 in FIG. 19 is secondary chamber 52. On the left hand side of barrier arrangement 60 in FIG. 19 is the void 66, with which the longitudinal perfusion channels 44 are in fluid communication. Thus, the cell chambers 30 are in indirect fluid communication with void 66 via the perfusion channels. In use of the module, barrier 64 is adapted as explained in more detail below, in order to provide a physiologically relevant blood-brain barrier model. This presents a barrier between cells in chambers 30 and compounds deliberately introduced into secondary chamber 52. In this way, the effect of candidate compounds in the blood stream on neural cells (for example) can be assessed, taking into account the blood-brain barrier.

Cells for use in the secondary chamber include cells which can form a blood brain barrier, gastro intestinal tract system, a reflex arc, a muscle skeletal system or other multi cell systems.

Neurons for example placed in chambers 30 can grow out through the longitudinally extending perfusion channels. A monolayer of cells may be formed on the left hand side surface of the barrier arrangement. A further monolayer of cells may be formed on the secondary chamber side of the barrier arrangement. The system can also be used to anchor skeletal cells.

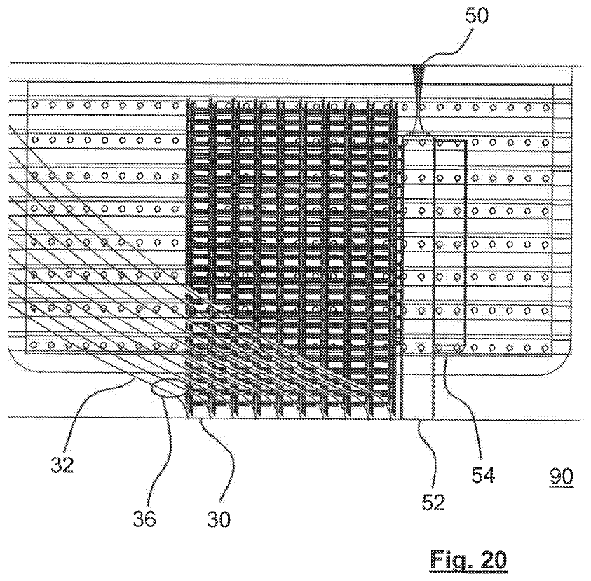

FIG. 20 shows a schematic longitudinal cross sectional view of part of the module. Here, cell passages 32 are shown as extended generally downwards in a sloping, curved arrangement, leading to cell chambers 30. In this embodiment, the base of chambers 30 is provided by substrate 90, described in more detail later.

As can be seen in FIG. 20, each cell introduction passage 32 has a generally decreasing diameter from its respective cell introduction port (not shown in FIG. 20), reaching a constricted region 36 just before opening into chamber 30. The constricted region 36 can be blocked, to prevent unwanted escape of cells in chamber 30, using a blocking element, described in more detail below.

FIG. 20 illustrates generally the array of perfusion channels associated with chambers 30. Additionally, secondary reservoir 54 and secondary chamber 52 are shown, along with secondary introduction port 50.

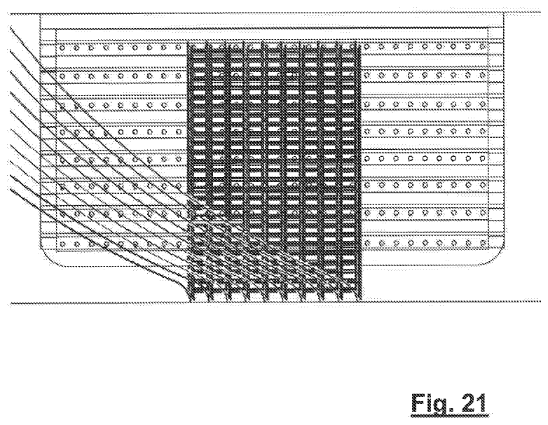

The view shown in FIG. 21 corresponds to that shown in FIG. 20, with the exception that the secondary cell introduction port 50, secondary chamber 52 and secondary reservoir 54 are not shown.

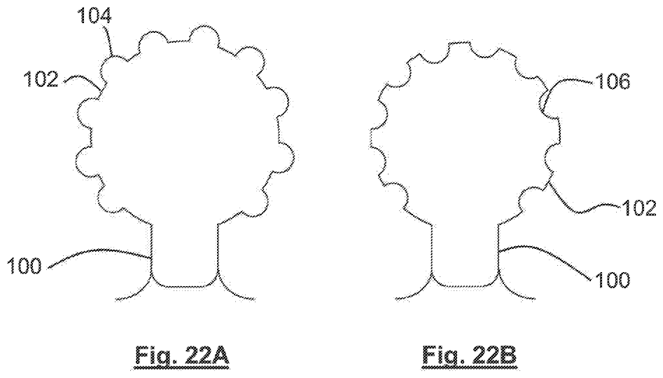

FIGS. 22A and 22B show schematic cross sectional views of suitable electrode structures for use with embodiments of the present disclosure. In FIG. 22A, the electrode structure has a generally spherical form, standing on columnar support 100. The sphere surface 102 has an array of rounded protrusions 104. In FIG. 22B, the electrode has a corresponding format, except that protrusions 104 are replaced by depressions 106. The effect of this is to provide additional surface area and surface features for interaction with cells which may provide electrogenic signals.

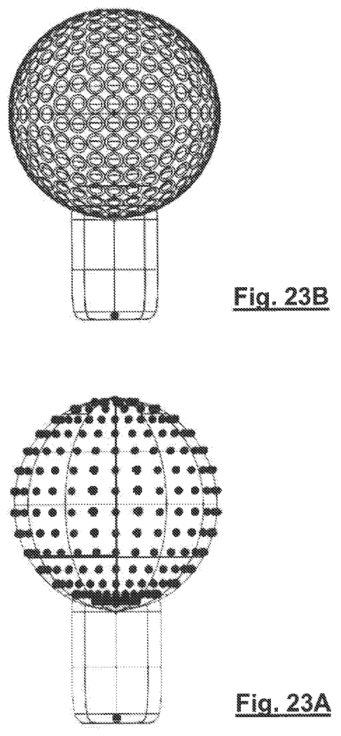

FIGS. 23A and 23B correspond to FIGS. 22A and 22B, showing front views of suitable electrode structures with protrusions or depressions, respectively.

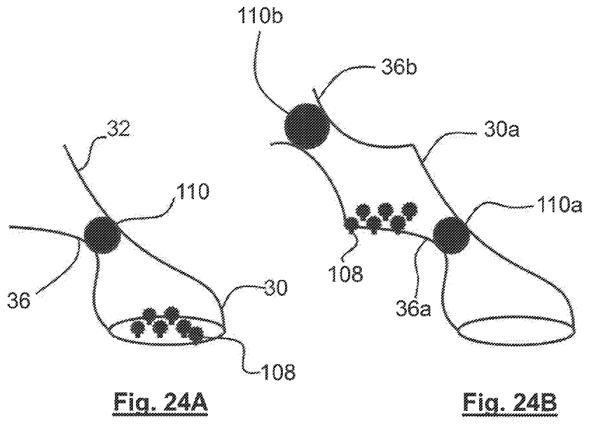

FIG. 24A shows a schematic view of chamber 30 in which electrodes 108 are located, the cell introduction passage 32 being blocked at constriction 36 by blocking bead 110. Blocking bead 110 can, for example, be a magnetic bead moved into position using an applied magnetic field. This blocks the exit route for cells in chamber 30.

A modified embodiment is shown in FIG. 24B, in which cell chamber 30a is provided by part of the cell introduction passage between two constrictions 36a and 36b. The cell chamber 30a once more being provided with electrodes 108. As shown in FIG. 24B, blocking bead 110a is located downstream along the cell introduction passage from chamber 30a and has a smaller diameter than blocking bead 110b located in constriction 36b upstream of chamber 30a. Constriction 36b has a greater diameter than constriction 36a, which permits blocking bead 110a to pass through constriction 36b to reach constriction 36a.

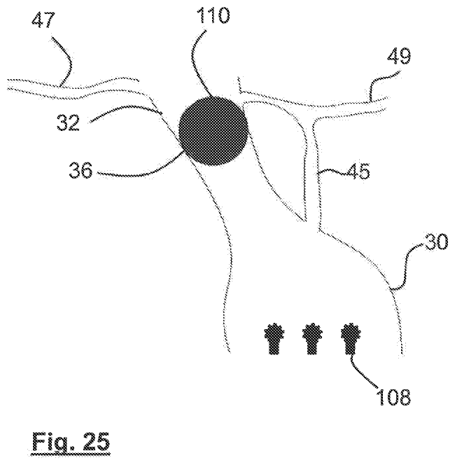

FIG. 25 shows cell chamber 30 with electrodes 108 and blocking bead 110 located at constriction 36 in cell passage 32. In FIG. 25, perfusion channels 45, 47, and 49 are shown. These do not have the regular, straight and orthogonal morphology shown in the embodiments described above. Instead, FIG. 25 is intended to show that the perfusion channels, and more generally the structure of the openings formed in the module, can be shaped in order more closely to model the physiological and environment seen by cells in vivo. Channels 45, 47 and 49 not only provide perfusion channels but also provide cell outgrowth channels, along which extra cellular structures can grow and extend. It will be understood that the required shape for the internal structures of the module can be designed by in vivo scanning of cells and their environments. The results of such scanning being used to reverse print the shapes encountered in vivo, in order to mimic the in vivo structure in the cell culture module.

In some case, a portion of a cell remains in a chamber and a portion of the cell extends outside the chamber such as extending into a cell outgrowth channel. For example, a cell nucleus may remain in a chamber and a cellular protrusion or extension may growth into an adjacent channel such as a cell outgrowth channel, a bypass channel, a perfusion channel, or others. A cellular protrusion may include a bleb, a ruffle, a filopodia, or a lamellipodia. A cellular protrusion may include a podosome. A cellular protrusion may include a pseudopodia extension. A cellular protrusion may include a dendrite, a dendrite spine, an axon, a growth cone, or combinations thereof.

A cell housed in one chamber may communicate with a cell housed in a different chamber by cell-cell communication. A cell housed in one chamber may communicate with at least two cells housed in two different chambers. A cell housed in one chamber may communicate with each cell housed in a plurality of different chambers of the cell culture module. Cell-cell communication may occur by paracrine signaling. For example, secretion of a chemokine or a cytokine from a cell that diffuses to a cell housed in an adjacent chamber. Cell-cell communication may occur by direct physical contact, for example, an adhesion contact or a cell junction. The cell culture module of the present disclosure permits for cellular protrusions such as axons to grow out from the chamber into a bypass channel or a perfusion channel or a combination thereof such that a synapse can form with a cell housed in an adjacent or distal chamber.

Cells are introduced into the chamber before the bead is added into the cell introduction passage. The beads are rolled in after the addition of cells. The locking of the beads in position verifies that the cells have reached the chambers.

The cells contact or at least partially engulf the electrodes and they grow out of the exits allowed. As discussed above, the exit topology may be a copy of growth pattern from vectors of known locations in vivo or they may be straight lines or curves.

The purpose and functioning of the module will now be described in more detail, with reference to a cell culture system of which the module is intended to form part.

The cell culture system includes a base unit (not shown) into which the cell culture module is releasably attachable. The cell culture system provides reservoirs for cell culture media to be provided to and extracted from the module. The cell culture system is controlled using a field programmable gate array (FPGA) of conventional type, which need not be described further here. The cell culture system provides for temperature sensing via temperature sensors, gas sensing (e.g. via O₂, CO₂, etc., sensors), glucose sensing and pH sensing. Suitable sensors will be well known to the skilled person and are not described further here. Rate-of-change (derivative) determination for these parameters is also envisaged. Temperature control is provided via the temperature sensors in combination with electrical heating elements, typically located so as to heat the cell culture media which is perfused into the module.

A sensor of the cell culture system may be a temperature sensor, a pH sensor, a gas sensor such as an O₂ sensor or CO₂ sensor, a glucose sensor, a level sensor, or any combination thereof. A sensor may be an optical sensor such as a bioluminescent sensor that senses a fluorescent or luminescent signal. A sensor may be an electrochemical sensor such as an amperometric sensor, a conductometric sensor, a potentiometric sensor, or a sensor that senses superficial charge. A sensor may be an opto-electric sensor such as a resonant mirror, a fiber optic, or a surface plasmon resonance sensor. A sensor may be a piezoelectric sensor such as a crystal resonance frequency sensor, a surface acoustic wave sensor, or a surface transverse wave sensor. A sensor may be a biosensor such as an enzyme electrode, an immunosensor, a DNA sensor, a microbial sensor, or any combination thereof. A cell culture system may have one or more sensors such as one or more electrochemical sensors, one or more optical sensors, one or more thermal sensors, one more resonant sensors, one or more ion-sensitive sensors, or any combination thereof. A cell culture system may have 1, 2, 3, 4, 5, 6, 7, 8, 9, 10, 11, 12, 13, 14, 15, 20, 30, 40, 50, 100, 200, 500 sensors or more. A cell culture system may have at least 1 sensor for each chamber of a cell culture system. A cell culture system may have at least 2 sensors for each chamber of a cell culture system. A cell culture system may have at least 3 sensors for each chamber of a cell culture system. A cell culture system may have at least 2, 3, 4, 5, 6, 7, 8 sensors or more for each chamber of a cell culture system.

In some embodiments, one or more analytes are added to one or more chambers of the cell culture system. The one or more analytes may or may not interact with an enzyme, an antibody, a protein, a nucleic acid, an antigen, a cell receptor, a cell, or a combination thereof present in the one or more chambers. The one or more sensors associated with the one or more chambers of the cell culture system sense a metric specific to that sensor. For example, a temperature sensor associated with a chamber may sense a temperature or a optical sensor associated with a chamber may sense a bioluminescence associated with a chamber.

A sensor may sense a metric specific to a single chamber. A sensor may sense one or more metrics of an array of chambers. A sensor may sense a metric of the module. A metric that a sensor may sense may include a temperature, an O₂ concentration, a CO₂ concentration, a pH level, a protein concentration, a bioluminescence concentration, a fluorescence concentration, a mass or weight, or any combination thereof.

A control system operatively coupled to a sensor may direct a routine sensing of a metric. For example, a control system may direct a thermistor to sense a temperature of a chamber every 60 minutes. Alternatively, a control system operatively coupled to a sensor may direct a specific sensing of a metric when a parameter is met. For example, a control system may direct a gas sensor to sense a CO₂ concentration of a chamber when a pH sensor senses a pH level of the chamber rises above 7.4. A user may input a routine sensing schedule into the control system. A user may input a specific sensing schedule into the control system.

A system of the disclosure may comprise a fluid delivery system. The fluid delivery system may comprise a pump for moving fluid within, to, and from one or more chambers of the cell culture module. A system may comprise one or more pumps. A system may comprise a pump for each of the plurality of chambers. A pump may be a positive displacement pump, an impulse pump, a velocity pump, a gravity pump, a steam pump, a vacuum pump, or a valveless pump.

A system of the disclosure may comprise a heating element. The heating element may provide heat to at least a portion of the cell culture module. A system may comprise one or more heating elements. A system may comprise one heating element for each chamber of the plurality of chambers. Each heating element may comprise an independent thermal zone, such that a temperature of each chamber of the plurality of chambers may be independently controlled by each heating element.

The module is intended to be replaceable and disposable. Using such an approach allows cells loaded in the module to be handled with ease, allowing the safe handover of cells and surrounding module without the need for a laminar flow cabinet or biosafety cabinet.

The FPGA controls the heaters such as strip heaters which may be aligned at two sides of a cell culture media conditioning chamber. A pH sensor is included in the closed loop, as well as an oxygen sensor, temperature sensor and a micro pump. With this closed loop system it is possible to deduce some factors from the pre media sensing and post sensing. For example, by measuring the pH pre media interaction with cells in superstructure and post pH measure after media interaction it is possible to get an indication of pH change due to reaction by-products. Furthermore, an understanding of the O2 or CO2 concentration pre-interaction and post-interaction with the cells gives an indication of the rate of metabolic activity in the system.

For the pre- and post-pH, oxygen, carbon dioxide and other parameter detection, a reservoir system is used in order to delay the fluid output or input, thereby yielding enough time and separation for measurements to occur.

The system is operable to maintain the temperature of the cells and other environmental parameters at normal physiological conditions. Additionally, the combination of devices in the system can be driven to mimic abnormal conditions within the body by changing pH, glucose levels, oxygen levels and so on.

It is intended that the module and system are used in one or more of the following applications:

Pre-clinical drug discovery.
II. Neuroscience and molecular biology research.
III. Toxicology studies.
IV. Field testing of contaminants.
V. Computational device for pattern recognition and embodied learning.
VI. Educational and research tool for artificial intelligence, computing engines/methods in natural systems and neuroengineering.
VII. A validation engine or platform for the development or test of neural implants (silicon, natural or modified living tissue which are constituted from multiple sources) in diseased or enhanced neural tissue in embodied and living systems (natural, cloned or otherwise).

The module and system fit with many application areas as outlined above, from preclinical drug discovery to security in field testing of toxins. The module and system can also be deployed for use as a computational device in pattern recognition or pattern computing. The module and system may be used as a platform for basic research in neural implants testing or as first step in designing embodiment systems i.e. biological tissue in silicon systems or 3D printed shells. The module and system find significant application in education as a teaching tool or research on basic functions of living systems.

Furthermore, the module and system are useful in drug development as a refinement device for post high throughput screening of drug candidates and pre in vivo test of compounds, biologics or other therapies. Computer scientists, engineers or physicists can use the module and system for modelling complex systems or learning in artificial systems. The module and system also a wide array of synthesized chemicals to be tested to assess their effect on human and/or animal health, i.e. to check for adverse effects or positive indications in human or animal systems.

The module and system can be deployed within larger autonomous system to chemical sites to testing for the presence of nerve agents for example and other contaminants in conflict zones, disasters area—natural or man-made—or other climes without risk to human lives. As such, this module and system find use with security personnel and military use in defense or humanitarian purposes.

A single cell specie or multiple cell species can be introduced into the module. The matrix of the module can be formed from a single base polymer or from a mix of several types of polymer. The type of cell configuration used may also vary. Single cells, organoid and slices, explants from animal embryonic tissue or explants from human biopsies can be introduced into the chambers. The device may use cells from human or animal origin, though the main focus of this disclosure is on human cells.

The module may be a disposable or non-disposable cartridge which fits within a larger base system. This module may be supplied with cells already introduced into the chambers. Alternatively, a user may introduce the cells. The cells can be introduced using simple pipettes or robotics systems.

As a drug development platform the module preferably contains a single cell specie or multiple cells connected in a physiologically realistic manner. For example in a co-culture system containing spinal motor neurons and muscle cells, the spinal motor neurons are able to cause the contraction of the muscle cells.

In the module, the cells are connected in one or both of the following ways: I. Functional coupling between cells i.e. a neuromuscular junction in vitro, neuron to neuron connection (residing in separate chambers), enteric nervous system to the gastrointestinal tract, the reflex arc in vitro motor neurons to muscle, dorsal root ganglia to muscle, layer topology in the CNS or brain and so on. II. Coupling via: a. Cells share similar media or grow in the same chamber or b. Fluid leaving one chamber is channeled to other cells making other cells come in contact with metabolites from the originating chamber.

The cells are separated by engineered substructures within a larger superstructure. These substructures serve as chambers which support further deposition of secondary polymers. The superstructure (monolithic matrix) itself is made of a primary polymer which lends itself to micro/nano structuring either by:

Light activated polymerization:

With single photon collimated light—lasers.

Multi photon light source or two photon absorption lithography.

Standard photo lithography with masking technologies similar to SU8

Micro machining with metallization

### Electrodeposition

In the secondary chamber of the module, for example, further substrate deposition may be carried out. For example a compliant polymer such as collagen or PA may be bound to an aminosilanized substrate. This allows for the use of contractile cells, which require a non-rigid substrate. This is of particular interest for the barrier arrangement. Other surface functionalization is envisaged, such as electro active polymers, allowing cells to be mechanically stimulated by an external modulating signal.

The electrodes in the chamber are used to detect action potential from neurons including excitatory post synaptic potentials. In contrast to known methods which utilize mushroom shaped electrodes to record from neurons which contact or at least partially engulf the electrodes, the approach used here and illustrated in FIGS. 22A, 22B, 23A and 23B increases the surface area available to the neurons and also promotes cell attachment. The general form of the electrode is a full spherical shape with hemispherical protrusions (FIGS. 22A and 23A) or hemispherical recesses (FIGS. 22B and 23B). These features further increase the area available for interfacing with a cell. The use of recesses instead of protrusions provides the increased surface area but at lower overall electrode volume. The height of the protrusions or the depth of the recesses can be modified as required, and the spacing of the protrusions or recesses can also be modified.

A cell culture module may comprise a glass, a silica, a silicon, a polymer, a hydrogel or any combination thereof. A polymer may comprise an elastomer, a thermoset, a thermoplastic, or any combination thereof. A polymer may comprise a polydimethylsiloxane (PDMS), poly(methylmethacrylate (PMMA)), polycarbonate (PC), polystyrene (PS), polyvinylchloride (PVC), polyethylene terephthalate (PET), or any combination thereof.

An electrode may comprise a metal. An electrode may comprise an alloy. An electrode may comprise aluminum, gold, lithium, copper, graphite, carbon, titanium, brass, silver, platinum, palladium, cesium carbonate, molybdenum (VI) oxide, or any combination thereof. An electrode may comprise a mixed metal oxide.

Modifying an electrode with a plurality of protrusions, a plurality of recesses, modifying by adding a surface roughness to the surface of an electrode may increase the surface area of the electrode. This modification or enhanced surface area may enhance the amount of cellular attachment to the electrode. This modification may enhance the portion of the electrode that is contacted or at least partially engulfed by a cell. This modification may enhance the portion of the electrode that is contacted by a cell. This modification may enhance an electrical connection between a cell and an electrode.

An electrode may comprise a spherical shape, a hemispherical shape, a mushroom shape, comprising a head portion and support portion, a rod-like shape, a cylindrical shape, a conical shape, a patch shape, or any combination thereof. A cell culture module may comprise electrodes having the same shape. For example, a module may comprise 10 electrodes of a mushroom shape. A cell culture module may comprise electrodes of more than one type of shape. For example, a module may comprise 10 electrodes of a mushroom shape and 10 electrodes of a conical shape.

An electrode may have a combination of one or more protrusions and one or more recesses. An electrode may have a combination of one or more protrusion shapes, such as a hemispherical protrusion, and one or more recess shapes, such as a hemispherical recess.

A protrusion may be a hemispherical protrusion. A protrusion may be a spike protrusion, a conical protrusion, a square or rectangular rod protrusion, an obelisk protrusion, a cylindrical protrusion, a hemispherical protrusion, or any combination thereof. A recess may be a hemispherical recess. A recess may be a V-groove recess, a dovetail recess, a spike recess, a conical recess, an cylindrical recess, a square or rectangular rod recess, a hemispherical recess, or any combination thereof.

An electrode may have one or more protrusions. An electrode may have 10 protrusions. An electrode may have at least 10 protrusions. An electrode may have 20 protrusions. An electrode may have at least 20 protrusions. An electrode may have 100 protrusions. An electrode may have at least 100 protrusions. An electrode may have 500 protrusions. An electrode may have at least 500 protrusions. An electrode may have 1000 protrusions. An electrode may have at least 1000 protrusions. An electrode may have 2000 protrusions. An electrode may have at least 2000 protrusions. An electrode may have 2, 3, 4, 5, 6, 7, 8, 9, 10, 15, 20, 25, 30, 35, 40, 45, 50, 100, 150, 200, 250, 500, 750, 1000, 1500, 2000, 3000, 4000, 5000 protrusions or more.

An electrode may have a surface concentration of protrusions of about 0.0001, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 2, 3, 4, 5, 6, 7, 8, 9, 10 protrusions per micrometer squared (pro/um²). An electrode may have a surface concentration of protrusions of about 0.0001 pro/um². An electrode may have a surface concentration of protrusions of about 0.001 pro/um². An electrode may have a surface concentration of protrusions of about 0.01 pro/um². An electrode may have a surface concentration of protrusions of about 0.1 pro/um². An electrode may have a surface concentration of protrusions of about 0.5 pro/um². An electrode may have a surface concentration of protrusions from about 0.0001 to about 0.01 pro/um². An electrode may have a surface concentration of protrusions from about 0.001 to about 0.01 pro/um². An electrode may have a surface concentration of protrusions from about 0.001 to about 0.1 pro/um². An electrode may have a surface concentration of protrusions from about 0.0005 to about 0.5 pro/um². An electrode may have a surface concentration of protrusions from about 0.05 to about 5 pro/um².

An electrode may have one or more recesses. An electrode may have 10 recesses. An electrode may have at least 10 recesses. An electrode may have 20 recesses. An electrode may have at least 20 recesses. An electrode may have 100 recesses. An electrode may have at least 100 recesses. An electrode may have 500 recesses. An electrode may have at least 500 recesses. An electrode may have 1000 recesses. An electrode may have at least 1000 recesses. An electrode may have 2000 recesses. An electrode may have at least 2000 recesses. An electrode may have 2, 3, 4, 5, 6, 7, 8, 9, 10, 15, 20, 25, 30, 35, 40, 45, 50, 100, 150, 200, 250, 500, 750, 1000, 1500, 2000, 3000, 4000, 5000 recesses or more.

An electrode may have a surface concentration of recesses of about 0.0001, 0.0005, 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 2, 3, 4, 5, 6, 7, 8, 9, 10 recesses per micrometer squared (rec/um²). An electrode may have a surface concentration of recesses of about 0.0001 rec/um². An electrode may have a surface concentration of recesses of about 0.001 rec/um². An electrode may have a surface concentration of recesses of about 0.01 rec/um². An electrode may have a surface concentration of recesses of about 0.1 rec/um². An electrode may have a surface concentration of recesses of about 0. 5rec/um². An electrode may have a surface concentration of recesses from about 0.0001 to about 0.01 rec/um². An electrode may have a surface concentration of recesses from about 0.001 to about 0.01 rec/um². An electrode may have a surface concentration of recesses from about 0.001 to about 0.1 rec/um². An electrode may have a surface concentration of recesses from about 0.0005 to about 0.5 rec/um². An electrode may have a surface concentration of recesses from about 0.05 to about 5 rec/um².

The surface of an electrode may be smooth. The surface of an electrode may have a surface roughness. A surface roughness may be uniform across the surface of an electrode. A portion of the surface of an electrode may have a surface roughness, such as a top portion of the electrode, a bottom portion of the electrode. An electrode may have alternating rows of smooth and rough portions.

A surface roughness may be about 5, 10, 15, 20, 25, 30, 35, 50, 100, 200, 300, 400, 500, 600, 700, 800, 900, 1000, 1500, 2000, 2500, 3000 nanometers (nm) or more. A surface roughness may be from about 5 to about 50 nm. A surface roughness may be from about 5 to about 100 nm. A surface roughness may be from about 5 to about 500 nm. A surface roughness may be from about 10 to about 50 nm. A surface roughness may be from about 10 to about 100 nm. A surface roughness may be from about 10 to about 500 nm.

A width of an electrode may be of a size to accommodate a cell to contact or at least partially engulf the electrode. A width of an electrode may be about 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 21, 22, 23, 24, 25, 26, 27, 28, 29, 30, 35, 40, 45, 50 micrometers (um). A width of an electrode may be about 2 um. A width of an electrode may be about 5 um. A width of an electrode may be about 10 um. A width of an electrode may be about 15 um. A width of an electrode may be about 20 um. A width of an electrode may be greater than 2 um. A width of an electrode may be greater than 3 um. A width of an electrode may be greater than 4 um. A width of an electrode may be greater than 5 um. A width of an electrode may be greater than 6 um. A width of an electrode may be greater than 7 um. A width of an electrode may be greater than 8 um. A width of an electrode may be greater than 9 um. A width of an electrode may be greater than 10 um. A width of an electrode may be a width of the support portion. A width of an electrode may be a width of the head portion.

An electrode may be operatively coupled to one or more electrodes. An electrode may be operatively coupled to at least two electrodes. An electrode may be operatively coupled to at least three electrodes. An electrode may be operatively coupled to each electrode of a plurality of electrodes of the cell culture module. An electrode may respond to a cellular output. An electrode may respond to an output from an electrode to which it is operatively coupled. An electrode response may include producing an output, such as an electrical signal.

The electrode structures are formed on a substrate e.g. glass. The substrate co-operates with the base of the monolith of the module in order to form the chambers.

The required shapes for the electrodes can be formed with multiphoton base material shaping and eventual metallization. Other processes e.g. PECVD, electrodeposition, e-beam lithography or associated techniques can also be used. For multiphoton lithography technology and 3D microprinting, commercially available devices that are known in the field of microfabrication can be used. The electrode shape may be realized with polymers, inverted metallization or by electrodeposition the polymer may then be removed by plasma etching.

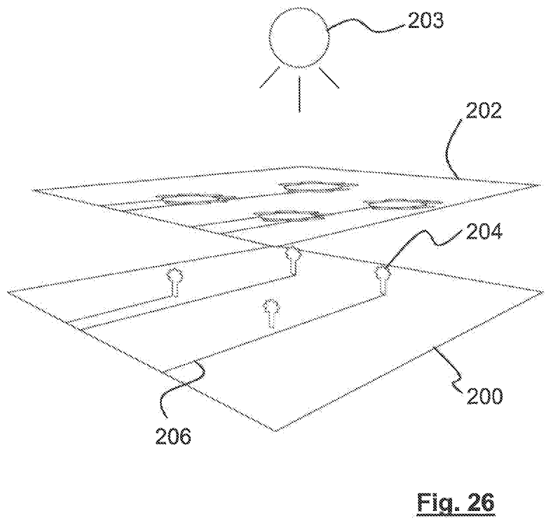

A suitable process is illustrated in FIGS. 26-30.

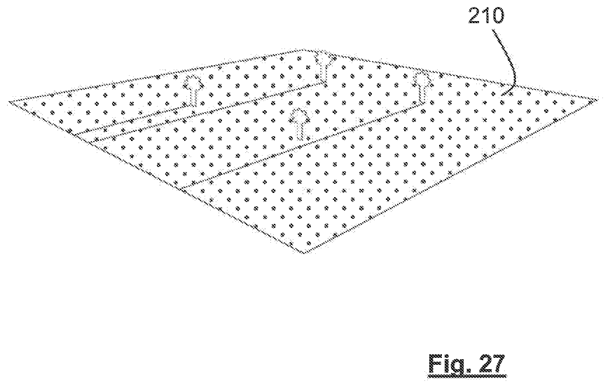

To develop the electrodes with attached conduction lines a glass substrate 200 already made with electrodes using DLW or two photon lithography is placed under a shadow mask 202. Reference number 203 identifies a lithography lamp. The mask 202 used to deposit a sacrificial layer 210 (shown in FIG. 27) leaving the electrodes 204 and conduction lines 206 exposed.

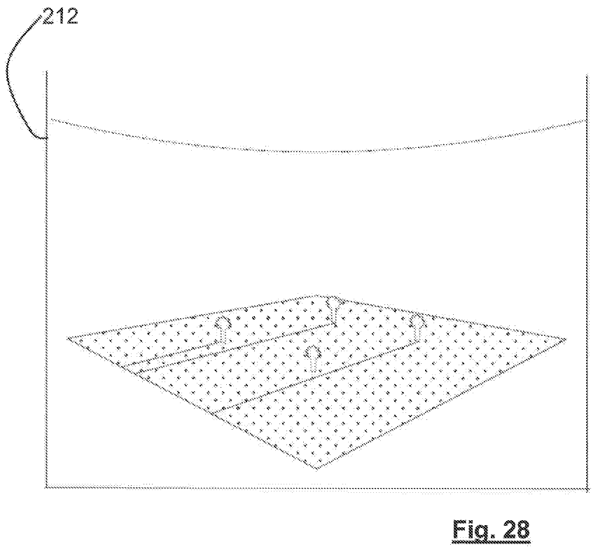

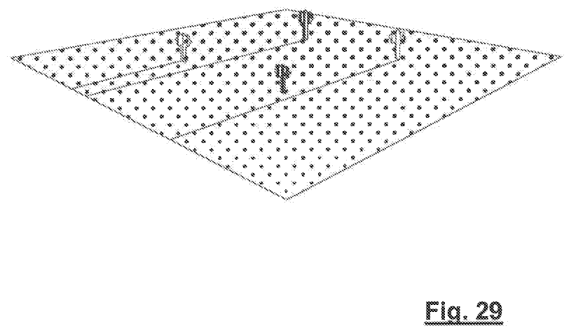

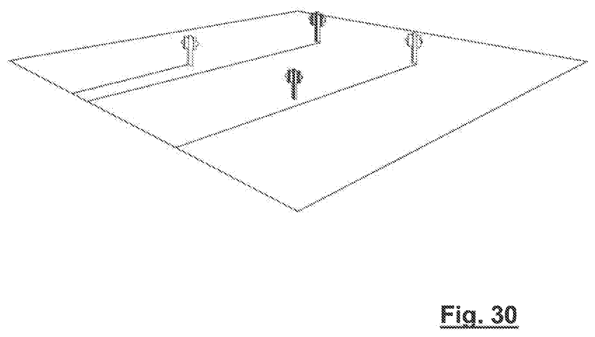

The exposed areas may be further developed using the process below, explained with reference to FIGS. 26-30:
I. A seed layer is used to coat the exposed electrodes e.g. at Cr (20 nm)/Au (200 nm)
II. Gold or platinum is then electrodeposited, as indicated in FIG. 28 which shows electroplate bath 212 containing the substrate with sacrificial layer. The gold or platinum is deposited at the electrodes, as shown in FIG. 29, to form the surface interfacing with living cells after further functionalization
III. The sacrificial layer is then stripped off with acetone, etching or other processes, to leave the seeded electrodes as shown in FIG. 30.

To promote the adhesion of the neurons (or other cells) to the electrodes, the topography of the electrode surface is modified to provide the protrusions or recesses discussed above. Additionally or alternatively, a controlled surface roughness is applied in the nanometer range, using further DLW or electrodeposition. Subsequently, the electrode surface is chemically functionalized with a poly-d-lysine and laminin sequence.

The matrix of the cell culture module is formed of a suitable polymer such as SU8 or other suitable biopolymer. In some embodiments, secondary polymers are adhered to parts of the matrix. Surface functionalization is carried out in order to promote the adhesion of the secondary polymers to the SU8 superstructure. The secondary polymers may be synthetic or natural polymers. This functionalization is done with a (3-aminopropyl)triethoxysilane (APTES) mix. The matrix is cleaned for 3 minutes in a plasma chamber at 100 W then soaked for 30 mins in 0.1M NaOH and allowed to dry at room temperature (22° C.). Required areas of the matrix are coated with (3-aminopropyl)trimethoxysilane for 4-5 minutes at room temperature before washing with excess distilled water. The matrix is incubated with a 0.5% glutaradehyde solution in phosphate buffered saline (PBS—176.8 mM NaCl, 2.7 mM KCl, 1.47 mM KH2PO4, 8.1 mM Na2HPO4, all from Fluka) solution for 30 mins. The matrix is cleaned with sterile PBS three times, removing the excess buffer. The secondary polymer is then filled into the desired area of the chip and allowed to dry/cure/fix for 30 mins before freezing to −10-20° C. for 1 hour. The polymer may be lyophilized for 6 hours at −20-40° C.

The module may be manufactured using multiphoton absorption lithography (or micro/nano 3D printing) which works because of cross linking (or other chemical processes) within micro/nano volumes or ‘voxels’. The methods required a polymer that is transparent at the working wavelength and creating a high intensity light at required vectors (i.e. in x, y and z). Therefore this method can create very fine arbitrary or regular structures. Suitable commercial manufacturing systems are based on scanning a laser wavefront within a substrate material. However, the speed of such systems may be limited by mechanical inertia of the scanning motors or ‘galvo scanners’. Systems which use scan-less methods to have the high light intensities at specified voxels may also be used, in order to increase the speed of direct laser writing of structures to a substrate.

The monolith is created by direct laser writing with either a scanning system or non scanning system. Using a polymer like SU8 for example the structure can withstand high aspect ratios dues to the inherent materials properties of SU8.

The electrodes may be formed on a glass substrate or a polymeric substrate, or on a composite substrate. Thereafter a mask is used to either cover the electrode or only the electrodes are exposed. The glass substrate is electroplated, therefore only the exposed electrodes are plated. The monolith is formed over the substrate.

The number of electrodes and the number of chambers is not particularly limited. Some edge electrodes may be used for example for verification and/or identification purposes. The resistance value of such electrodes may be intentionally modified so when the system is in use the system and/or module can be identified, whether locally or remotely.

Light guides are incorporated into the module, as illustrated in FIGS. 16 and 17. These are for optical interfacing with peripheral devices and/or for stimulation of optogenetic cells. The light guides may have lens structures integrated at their ends in order to allow light to be guided into the module via fibre optic cable or LEDs without causing significant noise in the underlying electronics or touching the electrode. The light avoids touching the electrodes to preclude the photoelectric effect.

The module has cell introduction passages with an inverted slanted funnel shape. This allows the introduction of single cells, re-aggregate cells, spheroids, micro organs, tissues or organoids into the chamber. As already described, the cell introduction passage is constricted at one or more locations. The diameter of the constriction is slightly larger than the cell body (or tissue, organoids, etc.) to be passed along the passage but smaller than the blocking bead that blocks it after the cell is introduced. The bead seals off the channel. Alternatively, multiple beads (as shown in FIG. 24B) can create levels of cells which can mimic in vivo tissue for example multiple levels in the cortex.

The beads serve two purposes. The first is to block to the chamber access, so as to allow, for example, only one cell per chamber. The bead may be selectively removed from its blocking configuration, thereby changing connectivity dynamics in the module. The second is that the bead can be provided with additional functionality. For example, the bead may provide gradual release of a chemical. Suitable chemicals can be released by time delay and/or by introducing a secondary chemical which releases the first. An example of this is glutamate and beads made of organic polymers.

Alternatively, release may be provided by heat activation, either globally or locally by using a laser wavefront for example. Alternatively, release may be provided by exposure to light of a given wavelength. The beads may carry immobilized proteins which may direct cells to perform actions. The beads may be activated by electric potential. Still further, chemicals released by the cell and/or electrogenic properties of the cell in the chamber may change the beads in terms of fluorescence color (wavelength) and/or rate of chemical release.

It is important to note the function of the bead may also be performed by a curable material such as a gel which can be deposited in the constriction. The bead or gel can be deposited with a micro placing device such as a delta robot to an arbitrary point in space by approach or location.

As illustrated in FIG. 25, the directionality of the perfusion/growth channel may be in any suitable direction.

Preferred embodiments of the disclosure incorporate a cell barrier system. Such a barrier is intended to replicate different aspects of in vivo like functions, such as:
Endothelial Cells e.g. blood brain barrier.
Musculature (Smooth, Skeletal or Cardiac)
Epithelial Cells e.g. lungs, uterus and respiratory
Connective Tissues e.g. skin, liver.
Other cell types.

The barrier is created in the superstructure in a net like grid. The grid is provided with an array of pores which are smaller than the cell type used in the barrier system. This part of the module is made using a DLW process and may have metalized parts to it, formed by electroplating.

Of particular interest in the preferred embodiments of the present disclosure is to replicate the blood brain barrier system. The barrier system achieves flexibility by proximity to other cells. The barrier is formed as a porous membrane as part of the monolithic matrix in a preferred embodiment, with cells seeded on one side.

### Computer Control Systems

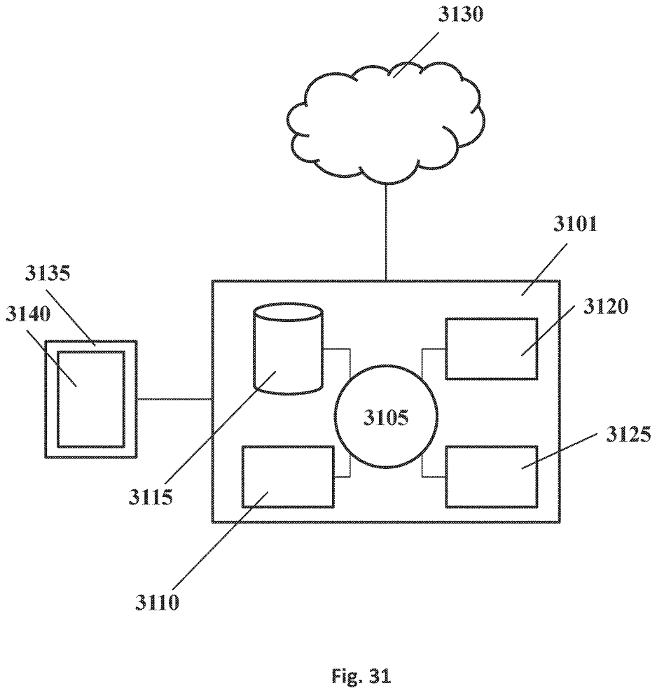

The present disclosure provides computer control systems that are programmed to implement methods of the disclosure. FIG. 31 shows a computer system 3101 that is programmed or otherwise configured to control the one or more sensors of the module, control one or more heating elements, one or more gas supplies (such as O2 or CO2), one or more microscopes, cameras (such as a CCD camera) associated with the module. The computer system 3101 can regulate various aspects of the cell culture system of the present disclosure, such as, for example, directing one or more sensors to make one or more measurements of a metric, interfacing with one or more heating elements, one or more gas supplies, one or more media sources to provide elements to the cell module system such as a regulated temperature, gas composition, and media supply. The computer system 3101 can be an electronic device of a user or a computer system that is remotely located with respect to the electronic device. The electronic device can be a mobile electronic device.

The computer system 3101 includes a central processing unit (CPU, also “processor” and “computer processor” herein) 3105, which can be a single core or multi core processor, or a plurality of processors for parallel processing. The computer system 3101 also includes memory or memory location 3110 (e.g., random-access memory, read-only memory, flash memory), electronic storage unit 3115 (e.g., hard disk), communication interface 3120 (e.g., network adapter) for communicating with one or more other systems, and peripheral devices 3125, such as cache, other memory, data storage and/or electronic display adapters. The memory 3110, storage unit 3115, interface 3120 and peripheral devices 3125 are in communication with the CPU 3105 through a communication bus (solid lines), such as a motherboard. The storage unit 3115 can be a data storage unit (or data repository) for storing data. The computer system 3101 can be operatively coupled to a computer network (“network”) 3130 with the aid of the communication interface 3120. The network 3130 can be the Internet, an internet and/or extranet, or an intranet and/or extranet that is in communication with the Internet. The network 3130 in some cases is a telecommunication and/or data network. The network 3130 can include one or more computer servers, which can enable distributed computing, such as cloud computing. The network 3130, in some cases with the aid of the computer system 3101, can implement a peer-to-peer network, which may enable devices coupled to the computer system 3101 to behave as a client or a server.

The CPU 3105 can execute a sequence of machine-readable instructions, which can be embodied in a program or software. The instructions may be stored in a memory location, such as the memory 3110. The instructions can be directed to the CPU 3105, which can subsequently program or otherwise configure the CPU 3105 to implement methods of the present disclosure. Examples of operations performed by the CPU 3105 can include fetch, decode, execute, and writeback.

The CPU 3105 can be part of a circuit, such as an integrated circuit. One or more other components of the system 3101 can be included in the circuit. In some cases, the circuit is an application specific integrated circuit (ASIC).

The storage unit 3115 can store files, such as drivers, libraries and saved programs. The storage unit 3115 can store user data, e.g., user preferences and user programs. The computer system 3101 in some cases can include one or more additional data storage units that are external to the computer system 3101, such as located on a remote server that is in communication with the computer system 3101 through an intranet or the Internet.

The computer system 3101 can communicate with one or more remote computer systems through the network 3130. For instance, the computer system 3101 can communicate with a remote computer system of a user (e.g., a cellular phone, a laptop computer, a tablet device, or any combination thereof). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 3101 via the network 3130.

Methods as described herein can be implemented by way of machine (e.g., computer processor) executable code stored on an electronic storage location of the computer system 3101, such as, for example, on the memory 3110 or electronic storage unit 3115. The machine executable or machine readable code can be provided in the form of software. During use, the code can be executed by the processor 3105. In some cases, the code can be retrieved from the storage unit 3115 and stored on the memory 3110 for ready access by the processor 3105. In some situations, the electronic storage unit 3115 can be precluded, and machine-executable instructions are stored on memory 3110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 3101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 3101 can include or be in communication with an electronic display 3135 that comprises a user interface (UI) 3140 for providing, for example, (a) a input provided by the user to maintain a temperature, a gas composition, a fluid composition, a fluid level, within one or more chambers of the cell culture system, (b) a input provided by the user to measure one or more metrics one or more times or at a specific time using the one or more sensors of the cell culture system, (c) a reminder or an alarm or a visual indicator provided to the user at the user interface when a sensor is not properly calibrated, when a measurement is complete, when a supply is deplete (such as a media supply or gas supply), or any combination thereof. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 3105. The algorithm can, for example, compare metrics measured by the one or more sensors to one or more reference metrics of a reference set to analyze a biological sample contained in the one or more chambers.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-20. (canceled)

21. A cell culture system comprising:
a transparent module having a plurality of chambers, a plurality of cells in each chamber attached to a substrate in each chamber, each chamber connected to a channel; and
a base unit comprising a control system and a fluid delivery system, the control system electrically connected to a light source and to an optical sensor configured for sensing a fluorescent or luminescent signal from one or more of the chambers, the fluid delivery system including a fluid reservoir, the base unit configured to retain the module and the fluid delivery system configured to deliver a fluid to each chamber through the channel connected to each chamber.

22. The cell culture system of claim 21 wherein the module is manufactured using a plurality of layers.

23. The cell culture system of claim 21 wherein the substrate comprises a porous membrane.

24. The cell culture system of claim 23 wherein the plurality of cells are seeded on one side of the porous membrane.

25. The cell culture system of claim 21 wherein the module has a width and/or a length less than about 2 inches.

26. The cell culture system of claim 21 wherein the module has 2 to 5 chambers.

27. The cell culture system of claim 21 wherein the module has 4 chambers.

28. The cell culture system of claim 21 wherein each chamber contains 100 or more cells.

29. The cell culture system of claim 21 wherein the fluid is a liquid.

30. The cell culture system of claim 21 wherein the module has a rectangular prism shape.

31. A cell culture system comprising:
a transparent cell culture module having a plurality of cell chambers;
a plurality of cells in each cell chamber;
each cell chamber connected to a channel;
a membrane structure positioned and configured to allow flow out of the cell chambers while containing the cells in the cell chambers; and
a control system and a fluid delivery system, the control system electrically connected to an optical sensor and to a light source, the fluid delivery system including a fluid reservoir connected to the channel for providing a fluid into the cell chambers.

32. The cell culture system of claim 31 wherein the membrane structure has a pore size of less than 0.05 micrometers.

33. The cell culture system of claim 31 wherein the module has a length and/or a width less than about 2 inches.

34. The cell culture system of claim 31 wherein the module has 2 to 5 chambers.

35. The cell culture system of claim 31 wherein the fluid is a cell medium liquid.

36. The cell culture system of claim 31 wherein the module has an rectangular prism shape.

37. The cell culture system of claim 31 further including a vacuum pump connected to the cell culture module.

38. A cell culture system comprising:
a quadrilateral transparent prism module having a plurality of cell chambers;
a plurality of cells in each cell chamber;
a plurality of fluid channels in the prism module, each fluid channel connecting into one of the cell chambers;
each cell chamber associated with a passageway in the prism module; and
a control system and a fluid delivery system, the control system electrically connected to an optical sensor and to a light source, the fluid delivery system including a fluid reservoir connected to the channel for providing a fluid into the cell chambers.

39. The cell culture system of claim 36 further including a membrane structure positioned and configured to allow flow out of the cell chambers while containing the cells in the cell chambers.

40. The cell culture system of claim 39 further including a vacuum pump connected to the prism module.
